(19) United States
(12) Patent Application Publication
(10) Pub. No.: US 2024/0374883 A1
(43) Pub. Date: Nov. 14, 2024

(54) MICRONEEDLE PATCH AND METHOD OF FABRICATION THEREOF

(71) Applicant: City University of Hong Kong, Kowloon (HK)

(72) Inventors: Chenjie Xu, Kowloon Tong (HK); Jinming Yu, Kowloon (HK); Yayi Zhao, Shatin (HK)

(21) Appl. No.: 18/660,303

(22) Filed: May 10, 2024

Related U.S. Application Data
(60) Provisional application No. 63/501,776, filed on May 12, 2023.

(51) Int. Cl. A61M 37/00; A61L 31/12; A61L 31/16

(52) U.S. Cl. CPC A61M 37/0015; A61L 31/129; A61L 31/16; A61L 2300/22; A61L 2400/12; A61M 2037/0023; A61M 2037/0046; A61M 2037/0053

(57) ABSTRACT

A microneedle patch includes a substrate and a plurality of microneedles disposed on the substrate, each of the plurality of microneedles including: (i) one or more polymers dissolvable in human skin layer, (ii) a plurality of lipid nanoparticles, and (iii) a nucleic acid drug encapsulated by the plurality of lipid nanoparticles; each of the plurality of microneedles being defined with a tip end portion that enables dermal penetration, and the microneedle patch being adapted to be stored at room temperature.

### FIELD OF INVENTION

The present invention relates to a microneedle patch and a method of fabrication thereof. In particular, the present invention relates to a microneedle patch for delivery of nucleic acid drug and a method of fabrication thereof.

### BACKGROUND OF INVENTION

Drugs are most often administered either orally or by parenteral injection. Oral drug delivery is the most convenient and patient-friendly drug administration method. However, first-pass elimination may reduce the bioavailability of orally ingested drugs. Direct delivery of drugs into the blood stream is achievable by hypodermic injection but it requires professional help to administer an injection. To mitigate these problems, microneedle-mediated drug delivery system is available for almost painless self-administration of therapeutic drugs.

Dissolvable microneedle is polymeric, microscopic needles that encapsulates pharmaceuticals within their matrix. Upon insertion of the dissolvable microneedle into the skin, the degradation of the polymeric compound is initiated to thereby release the drug for systemic or local delivery.

Nucleic acid drugs provide novel therapeutic modalities with characteristics that differ from those of small molecules and antibodies. Nucleic acid drugs are the latest version of medicine and attract great interest from academia and industry. They are useful in treating human diseases such as cancers, viral infections, and genetic disorders due to unique characteristics that make it possible to approach targets protein that is not pharmacologically capable of being targeted. Nucleic acid drugs can be designed regardless of the localization or structure of the target molecule. Once a platform is established, rapid and efficient drug development can be expected as it becomes possible to create a drug simply by changing the nucleotide sequence of the target gene. The success of COVID-19 mRNA vaccine is a clear demonstration of the potentials of this type of drugs.

A problem with nucleic acid drug would be the temperature sensitivity. Nucleic acids are relatively unstable in room temperature and are hard to enter the cells post the administration. While nucleic acids can be delivered through intravenous injection, intramuscular injection, subcutaneous injection, intradermal and transdermal injection is ideal for vaccination and treating some local diseases (e.g. skin diseases). Nucleic acid drugs are the latest version of medicine and attract great interest from academia and industry.

The invention seeks to eliminate or at least to mitigate some of the above-mentioned shortcomings of the prior art.

### SUMMARY OF INVENTION

In a first aspect, there is provided a microneedle patch comprising a substrate and a plurality of microneedles disposed on the substrate, wherein each of the plurality of microneedles comprises: (i) one or more polymers dissolvable in human skin layer, (ii) a plurality of lipid nanoparticles, and (iii) a nucleic acid drug encapsulated by the plurality of lipid nanoparticles; wherein each of the plurality of microneedles is defined with a tip end portion that enables dermal penetration, and wherein the microneedle patch is adapted to be stored at room temperature (0-40° C.).

Preferably, the nucleic acid drug is selected from a group consisting of plasmid DNA (pDNA), minicircular DNA (mcDNA), messenger RNA (mRNA), microRNA (miRNA), antisense oligonucleotides (ASOs), small interfering RNA (siRNA), aptamers, and ribozymes.

Preferably, the plurality of lipid nanoparticles are formed from at least a component selected from a group consisting of (4-hydroxybutyl) azanediyl bis(hexane-6,1-diyl)bis(2-hexyldecanoate), 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), 2,3-dioleyloxy-N-[2-(sperminecarboxamido)ethyl]-N,N-dimethyl-1-propanaminium trifluoroacetate (DOSPA), (2S)-2,5-bis(3-aminopropylamino)-N-[2-(dioctadecylamino)acetyl]pentanamide (DOGS; Transfectam), N1-[2-((1 S)-1-[(3-aminopropyl)amino]-4-[di(3-aminopropyl)amino]butylcarboxamido)ethyl]-3,4-di[oleyloxy]-benzamide (MVL5), DC-Cholesterol59, N4-cholesteryl-spermine (GL67), (6Z,9Z,28Z,31Z)-heptatriaconta-6,9,28,31-tetraen-19-yl 4-(dimethylamino) butanoate (DLin-MC3-DMA; MC3), di((Z)-non-2-en-1-yl) 9-((4-(dimethylamino)butanoyl)oxy)heptadecanedioate (L319), biodegradable lipids heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102))78 and ((4-hydroxybutyl)azanediyl)bis(hexane-6,1-diyl)bis(2-hexyldecanoate) (ALC-0315), 3,6-bis(4-(bis(2-hydroxydodecyl)amino)butyl)piperazine-2,5-dione (cKK-E12), (2-hexyldecanoate), 2-[(polyethylene glycol)-2000]-N,N-ditetradecylacetamide, 1,2-dimyristoyl-rac-glycero-3-methoxypolyethylene glycol-2000 (PEG2000-DMG), 1,2-distearoyl-rac-glycero-3-methoxypolyethylene glycol-2000 (PEG2000-DSG), 1,2-distearoyl-sn-glycero-3-phosphoethanolamine-N-[methoxy(polyethylene glycol)-2000] (DSPE-PEG(2000)) 1,2-distearoyl-sn-glycero-3-phosphocholine (DSPC), 1,2-dioleoyl-sn-glycero-3-phosphoethanolamine (DOPE), Dipalmitoylphosphatidylcholine (DPPC), N-bis(2-hydroxyethyl)-N-methylethan-1-aminium bromide (BHEM-Cholesterol), and 3β-[N-(N′,N′-dimethylaminoethane)-carbamoyl]cholesterol (DC-Cholesterol).

Preferably, the lipid nanoparticles are formed from an ionizable cationic lipid, a polyethylene glycol (PEG), a cholesterol, and a helper lipid.

Preferably, the ionizable cationic lipid is 1,2-dioleoyl-3-trimethylammonium-propane (DOTAP).

Preferably, the polyethylene glycol (PEG) is 1,2-distearoyl-sn-glycero-3-phosphoethanolamine-N-[methoxy-(polyethylene glycol)-2000] (DSPE-PEG(2000)).

Preferably, the helper lipid is Dipalmitoylphosphatidylcholine (DPPC).

Preferably, the lipid nanoparticles are 50-220 nm or 100-150 nm in size.

Preferably, the lipid nanoparticles are 120±30 nm in size, with size distribution (PDI) less than 0.2.

Preferably, the one or more polymers are selected from a group comprising polyvinylpyrrolidone, polyvinyl alcohol, hyaluronic acid, collagen, polyethylene glycol, their derivatives or a mixture thereof.

Preferably, the one or more polymers is a polymer mixture of polyvinylpyrrolidone and polyvinyl alcohol.

Preferably, the polyvinylpyrrolidone is in a ratio of 0:100 to 100:0(%) against the polyvinyl alcohol.

Preferably, the polyvinylpyrrolidone has a molecular weight of 23000-32000 or 35000-51000 Daltons.

Preferably, the polyvinyl alcohol has a molecular weight of 72600-81400, 81400-94600 or 17600-26400 Daltons.

Preferably, the polymer mixture is further mixed with a sugar selected from a group consisting of sucrose, trehalose, dextrose and a mixture thereof to form a further mixture, and wherein the sugar has a concentration of 0.1-10% (w/w) in the further mixture.

Preferably, the nucleic acid drug is adapted to retain more than 50% of original activity in a sealed centrifuge tube at room temperature (0-40° C.) for a period of at least 42 days, and remains biologically active for at least 60 days.

The nucleic acid drug is adapted to retain more than 50% of original activity for at least 6 months at a storage temperature of 0-40° C. and humidity of or below about 40%.

In a second aspect, there is provided a method of fabricating a microneedle patch, comprising steps of: (i) forming a plurality of nanoparticles with a nucleic acid drug encapsulated therein, (ii) mixing the plurality of nanoparticles with a polymeric solution to form a matrix solution, wherein the polymeric solution comprises one or more polymers dissolvable in human skin layer, (iii) casting the matrix solution in a mold defined with a plurality of recesses for producing microneedle structures, wherein each of the microneedle structure is defined with a tip end portion that enables dermal penetration, (iv) drying the matrix solution to form a microneedle patch storable in room temperature (0-40° C.), and (v) removing the microneedle patch from the mold.

Preferably, the plurality of nanoparticles is formed by steps of: (i) dissolving the at least one lipid component in a first solvent and the nucleic acid in a second solvent, (ii) mixing the dissolved lipid component with the dissolved nucleic acid drug to form a drug mixture, (iii) passing the drug mixture though both organic and aqueous phases to form nanosuspensions, (iv) dialysing the nanosuspensions to remove the first and second solvents from the drug mixture, and (v) centrifuging the nanosuspensions in a buffer solution to form the plurality of nanoparticles.

Preferably, the first solvent is ethanol.

Preferably, the second solvent is sodium citrate buffer solution.

Preferably, the buffer solution is phosphate buffered saline solution.

Preferably, the drug mixture is passed though the organic and aqueous phases at a flow rate of 1:3.

Preferably, the drug mixture has a total flow rate of 4-6 ml/min between the aqueous and organic phases.

Preferably, the drug mixture has a lipid concentration of 3-14 mg/ml.

Preferably, the drug mixture is passed though the organic and aqueous phases in a microfluid chip, and wherein the microfluid chip is a Y-type or a T-type microfluidic chip.

Preferably, the dialysing of the nanosuspensions is performed at a low temperature for 24 to 48 hours, and preferably the low temperature is 4° C.

Preferably, the polymeric solution has a concentration of 20-30%.

Preferably, the mold is treated with plasma before the matrix solution is cast therein.

Preferably, the method further comprising a step of packing the microneedle patch in a nitrogen (N₂) environment.

Preferably, the method further comprising a step of storing the microneedle patch under temperature between 0-40° C. and humidity of or below about 40%.

Preferably, the method further comprising a step of storing the microneedle patch in a dry box at room temperature.

Preferably, the mold is a PDMS mold or a metal mold or a resin mold.

Preferably, the matrix solution is dried at a temperature between 25-30° C.

Preferably, the nucleic acid drug is selected from a group consisting of plasmid DNA (pDNA), minicircular DNA (mcDNA), messenger RNA (mRNA), microRNA (miRNA), antisense oligonucleotides (ASOs), small interfering RNA (siRNA), aptamers, and ribozymes.

Preferably, the plurality of lipid nanoparticles are formed from at least a component selected from a group consisting of (4-hydroxybutyl) azanediyl bis(hexane-6,1-diyl)bis(2-hexyldecanoate), 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), 2,3-dioleyloxy-N-[2-(sperminecarboxamido)ethyl]-N,N-dimethyl-1-propanaminium trifluoroacetate (DOSPA), (2S)-2,5-bis(3-aminopropylamino)-N-[2-(dioctadecylamino)acetyl]pentanamide (DOGS; Transfectam), N1-[2-((1 S)-1-[(3-aminopropyl)amino]-4-[di(3-aminopropyl)amino]butylcarboxamido)ethyl]-3,4-di[oleyloxy]-benzamide (MVL5), DC-Cholesterol59, N4-cholesteryl-spermine (GL67), (6Z,9Z,28Z,31Z)-heptatriaconta-6,9,28,31-tetraen-19-yl 4-(dimethylamino) butanoate (DLin-MC3-DMA; MC3), di((Z)-non-2-en-1-yl) 9-((4-(dimethylamino)butanoyl)oxy)heptadecanedioate (L319), biodegradable lipids heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102))78 and ((4-hydroxybutyl)azanediyl)bis(hexane-6,1-diyl)bis(2-hexyldecanoate) (ALC-0315), 3,6-bis(4-(bis(2-hydroxydodecyl)amino)butyl)piperazine-2,5-dione (cKK-E12), (2-hexyldecanoate), 2-[(polyethylene glycol)-2000]-N,N-ditetradecylacetamide, 1,2-dimyristoyl-rac-glycero-3-methoxypolyethylene glycol-2000 (PEG2000-DMG), 1,2-distearoyl-rac-glycero-3-methoxypolyethylene glycol-2000 (PEG2000-DSG), 1,2-distearoyl-sn-glycero-3-phosphoethanolamine-N-[methoxy(polyethylene glycol)-2000] (DSPE-PEG(2000)) 1,2-distearoyl-sn-glycero-3-phosphocholine (DSPC), 1,2-dioleoyl-sn-glycero-3-phosphoethanolamine (DOPE), Dipalmitoylphosphatidylcholine (DPPC), N-bis(2-hydroxyethyl)-N-methylethan-1-aminium bromide (BHEM-Cholesterol), and 3β-[N-(N′,N′-dimethylaminoethane)-carbamoyl]cholesterol (DC-Cholesterol).

Preferably, the lipid nanoparticles are formed from an ionizable cationic lipid, a polyethylene glycol (PEG), a cholesterol, and a helper lipid.

Preferably, the ionizable cationic lipid is 1,2-dioleoyl-3-trimethylammonium-propane (DOTAP).

Preferably, the polyethylene glycol (PEG) is 1,2-distearoyl-sn-glycero-3-phosphoethanolamine-N-[methoxy-(polyethylene glycol)-2000] (DSPE-PEG(2000)).

Preferably, the helper lipid is Dipalmitoylphosphatidylcholine (DPPC).

Preferably, the lipid nanoparticles are 50-220 nm or 100-150 nm in size.

Preferably, the lipid nanoparticles are 120±30 nm in size, with size distribution (PDI) less than 0.2.

Preferably, the one or more polymers are selected from a group comprising polyvinylpyrrolidone, polyvinyl alcohol, hyaluronic acid, collagen, polyethylene glycol, their derivatives or a mixture thereof.

Preferably, the one or more polymers is a polymer mixture of polyvinylpyrrolidone and polyvinyl alcohol.

Preferably, the polyvinylpyrrolidone is in a ratio of 0:100 to 100:0(%) against the polyvinyl alcohol.

Preferably, the polyvinylpyrrolidone has a molecular weight of 23000-32000 or 35000-51000 Daltons.

Preferably, the polyvinyl alcohol has a molecular weight of 72600-81400, 81400-94600 or 17600-26400 Daltons.

Preferably, the methods further comprising a step of mixing the polymer mixture with a sugar selected from a group consisting of sucrose, trehalose, dextrose and a mixture thereof to form a further mixture, and wherein the sugar has a concentration of 0.1-10% (w/w) in the further mixture.

Preferably, the nucleic acid drug is adapted to retain more than 50% of original activity in a sealed centrifuge tube at room temperature (0-40° C.) for a period of at least 42 days, and remains biologically active for at least 60 days.

The nucleic acid drug is adapted to retain more than 50% of original activity for at least 6 months at a storage temperature of 0-40° C. and humidity of or below about 40%.

### BRIEF DESCRIPTION OF FIGURES

In order that a more precise understanding of the above-recited invention can be obtained, a more particular description of the invention briefly described above will be rendered by reference to specific embodiments thereof that are illustrated in the appended drawings. The drawings presented herein may not be drawn in scale and any reference to dimensions in the drawings or the following description is specific to the embodiments disclosed.

FIG. 1, part a, shows an embodiment of a method of fabrication of a microneedle patch according to the present invention; FIG. 1, part b, shows a fluorescence microscopy image of a first embodiment of a microneedle patch (FITC-LNP-MN) of the present invention; FIG. 1, part c, shows a photograph of the microneedle patch of part b; and FIG. 1, part d, shows a photograph of a second embodiment of a microneedle patch (mRNA-LNP) according to the present invention;

FIG. 2, part a, shows a cross-section diagram of a lipid nanoparticle, and the molecular formulae of the four components of the lipid nanoparticles within a microneedle patch, according to an embodiment of the present invention; FIG. 2, part b, shows a photograph of a FITC-dextran-LNP solution; and FIG. 2, part c, illustrates a microfluidic process of forming a plurality of lipid nanoparticles encapsulated with the nucleic acid drug;

FIG. 3 shows a table of components from which lipid nanoparticles are formed;

FIG. 4 demonstrates an optimising process of forming a microneedle patch, by utilising Design of Experiments (DOE);

FIG. 5, part a, provides a response surface plot; FIG. 5, part b, provides a two-dimensional (2D) contour plot of DOE for the optimisation of lipid nanoparticle sizes with total flow rate (TFR) versus lipid concentration; FIG. 5, part c, shows a response surface plot; and FIG. 5, part d, shows a 2D contour plot of DOE for the optimisation of lipid nanoparticle polydispersity index (PDI) with TFR versus lipid concentration;

FIG. 6, part a, is a graph showing the hydrodynamic diameters of mRNA-LNPs and FITC-Dextran-LNPs; FIG. 6, parts b and c, show transmission electron microscopy (TEM) images of mRNA-LNPs and FITC-Dextran-LNPs; and FIG. 6, part d, shows the quantification and measurement of hydrodynamic diameters and zero potentials of mRNA-LNPs and FITC-Dextran-LNPs;

FIGS. 7a-7c show the fluorescence images of mesenchymal stem cells (MSCs) transfected with a) enhanced green fluorescent protein (EGFP) mRNA alone, b) EGFP mRNA-lipofectamine and c) EGFP mRNA-LNPs;

FIG. 7d demonstrates the quantification of average fluorescence per fluorescent cell in the fluorescence images of FIGS. 7a-7c;

FIGS. 7e-7g show the flow cytometry analysis of MSCs transfected with e) EGFP mRNA alone, f) EGFP mRNA-lipofectamine and g) EGFP mRNA-LNPs;

FIG. 7h shows the quantification of fluorescence intensity of cells in the flow cytometry analysis of FIGS. 7e-7g;

FIG. 8a-8c show the fluorescence images of MSCs transfected with a) FITC-dextran alone, b) FITC-dextran-lipofectamine in microneedles and c) FITC-dextran-LNP-MN;

FIG. 8d demonstrates the quantification of average fluorescence per fluorescent cell in the fluorescence images of FIGS. 8a-8c;

FIGS. 8e-8g show the flow cytometry analysis of MSCs transfected with e). FITC-dextran alone in MN, f) FITC-dextran-lipofectamine in microneedles and g) FITC-dextran-LNP-MN;

FIG. 8h shows the quantification of fluorescence intensity of cells in the flow cytometry analysis of FIGS. 8e-8g;

FIG. 9 is a graph showing the relationship between the load force and the tip displacement of LNP-MN with different concentrations (30%, 25%, 20%, 15%, 10%) of PVP under drying conditions at 20° C., 40° C. and 60° C.;

FIG. 10, part a, shows a response surface plot; FIG. 10, part b, shows a 2D contour plots of DOE for the polyvinylpyrrolidone (PVP) concentration and drying temperature on dispersibility; FIG. 10, part c, shows a response surface plot; and FIG. 10, part d, shows a 2D contour plots of Young's Modulus for PVP concentration and drying temperature;

FIGS. 11a-11c show the fluorescence images of MSCs transfected with a) EGFP mRNA alone, b) EGFP mRNA-lipofectamine and c) EGFP mRNA-LNP-MN;

FIG. 11d demonstrates the quantification of average fluorescence per fluorescent cell in the fluorescence images of FIGS. 11a-11c;

FIGS. 11e-11g show the flow cytometry analysis of MSCs transfected with e) EGFP mRNA alone, f) EGFP mRNA-lipofectamine and g) EGFP mRNA-LNP-MNs;

FIG. 11h shows the quantification of fluorescence intensity of cells in the flow cytometry analysis of FIGS. 11e-11g;

FIGS. 11i-11k illustrate the transfection results of FIGS. 11e-11g after 42 days of storage;

FIG. 11l shows the quantification of fluorescence intensity of cells in the flow cytometry analysis of FIGS. 11i-11k;

FIGS. 11m-11o illustrate the transfection results of FIGS. 11e-11g after 60 days of storage;

FIG. 11p shows the quantification of fluorescence intensity of cells in the flow cytometry analysis of FIGS. 11m-11o;

FIGS. 12a-12c show the fluorescence images of MSCs transfected with a) FITC-dextran alone, b) FITC-dextran-lipofectamine in microneedles and c) FITC-dextran-LNP-MN;

FIG. 12d demonstrates the quantification of average fluorescence per fluorescent cell in the fluorescence images of Figures of 12a-12c;

FIGS. 12e-12g show the flow cytometry analysis of MSCs transfected with e) FITC-dextran alone in MN, f) FITC-dextran-lipofectamine in microneedles and g) FITC-dextran-LNP-MN;

FIG. 12h shows the quantification of fluorescence intensity of cells in the flow cytometry of FIGS. 12e-12g;

FIG. 13, part a, shows the fluorescence image of fibroblast cells transfected with FITC-dextran-LNP-MN fabricated by 25% of PVP after drying at 60° C.; FIG. 13, part b, shows the fluorescence image of fibroblast cells transfected with FITC-dextran-LNP-MN fabricated by 30% of PVP after drying at 60° C.; FIG. 13, part c, shows the fluorescence image of fibroblast cells transfected with FITC-dextran alone; and

FIG. 14, part a, shows the fluorescence images of fibroblast cells transfected with FITC-dextran alone; part b shows the fluorescence images of fibroblast cells transfected with FITC-dextran-lipofectamine; part c shows the fluorescence images of fibroblast cells transfected with FITC-dextran-LNPs; part d shows the fluorescence images of HEK 293 cells transfected with FITC-dextran alone; part e shows the fluorescence images of HEK 293 cells transfected with FITC-dextran-lipofectamine; and part f shows the fluorescence images of HEK 293 cells transfected with FITC-dextran-LNPs;

FIG. 15a is a table demonstrating the DOE data for lipid nanoparticles;

FIG. 15b is a table demonstrating the DOE data for LNP-MNs;

FIG. 16a demonstrates the intradermal delivery of mRNA to mice using an embodiment of a microneedle patch (i.e. EGFP mRNA-LNP-MNs) according to the present invention.

FIG. 16b shows heat maps of mice that have received the intradermal delivery of mRNA of FIG. 16a.

### DETAILED DESCRIPTION OF PREFERRED EMBODIMENTS

### 1. Overview of the Present Invention

Nucleic acid drugs are a class of DNA/RNA molecules and are considered capable of treating protein-related diseases through regulating target proteins expression. A number of nucleic acid drugs have been approved for treating various diseases caused by genetic defects, viral infections and genetic mutations. The wide usage of mRNA vaccines in the COVID-19 pandemic has demonstrated the safety and great potentials of these drugs.

This invention describes a microneedle patch dissolvable in skin layer post skin penetration for intradermal delivery of nucleic acid drugs. The microneedle patch can be stored and distributed in room temperature (0-40° C.). The microneedle patch of the present invention involves the development of lipid nanoparticles for the encapsulation of nucleic acid drugs and the subsequent fabrication of microneedles using these lipid nanoparticles. Size and components of the lipid nanoparticles are systematically optimized to maximize the stability of nucleic acids during the storage in room temperature (0-40° C.) and dry state. The microneedles are manufactured using polymers that dissolve in the skin layer post the skin penetration. The polymers also have minimal change to the stability of nucleic acids.

The dissolvable microneedle is suitable for intradermal and transdermal delivery of nucleic acid drugs such as mRNA vaccine, DNA vaccine, and siRNA drugs etc. In an embodiment, the nucleic acid drug is selected from a group consisting of plasmid DNA (pDNA), minicircular DNA (mcDNA), messenger RNA (mRNA), microRNA (miRNA), antisense oligonucleotides (ASOs), small interfering RNA (siRNA), aptamers, and ribozymes. The dissolvable microneedle can be stored and distributed in room temperature (0-40° C.).

The dissolvable microneedle allows the pre-packaging of nucleic acid in microneedle formulation, which can be topically applied. The dissolvable microneedle also allows rapid or fast drug administration and large-scale vaccination and treating local diseases. In addition, the lipid nanoparticles help the cellular entrance of the nucleic acids. The dissolvable microneedle can be kept and distributed in in room temperature (0-40° C.), which would low down the total cost of drugs to the patients, thereby eliminating the challenges and costs associated with cold storage and transportation.

The dissolvable microneedle forms a patch with a base supporting an array of needles; a substrate to which the base is formed with; and a drug. The drug is a nucleic acid drug. The dissolvable microneedle dissolves after penetration of skin at a dissolving duration of between about 1 seconds to about 10 mins. At least part of the needle detaches from the base when dissolved. More preferably, the needle dissolves regardless of a condition selected from a group consisting temperature, humidity, skin quality, age, sex, and race.

In the present invention, size of the lipid nanoparticles is controlled between 50-220 nm or 100-150 nm. The purified lipid nanoparticles are then mixed with the aqueous solution of polyvinylpyrrolidone (PVP) and Polyvinyl alcohol (PVA) and sugars. The size (molecular weight) of PVP can be 23000-32000 or 35000-51000 Daltons. The size (molecular weight) of PVA can be 72600-81400, 81400-94600 or 17600-26400 Daltons. The ratio ranges of PVP and PVA is from 0:100 to 100:0(%). The sugars could be sucrose or trehalose or dextrose or their mixtures. The concentration of sugars in the PVP/PVA/sugar mixture is 0.1-10% (w/w).

### 2. Fabrication of Dissolvable Microneedles

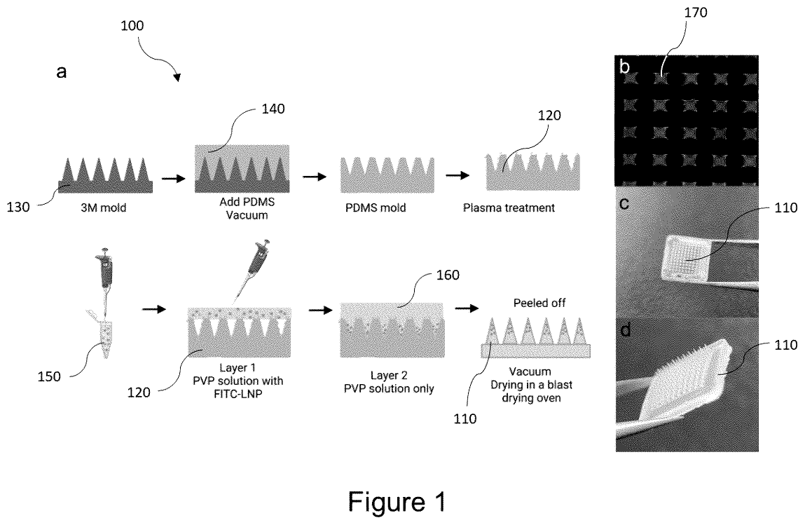

Referring now to FIG. 1a, which shows an embodiment of a method 100 for preparing microneedle patches 110 according to the present invention. Prior to forming the microneedle patches 110, preparation of a microneedle mold 120 is required. Preferably, the microneedle mold 120 is formed with polydimethylsiloxane (PDMS). In another embodiment, other biocompatible molding materials 140 such as certain types of metal and resin may also be used to form the microneedle mold 120.

FIG. 1a shows the use of a master mold 130 to create a microneedle mold 120 for manufacturing microneedle patches 110. The master mold 130 has a pattern of needle-like structures. Molding material 140 is poured onto the master mold 130 and set under vacuum to form a microneedle mold 120, which is a replica of the opposite of the master mold 130, with an array of needle-like recesses. The surface of microneedle mold 120 is then treated with plasma.

The plasma treatment to the surface of the microneedle mold 120 aims at enhancing its hydrophilicity. The mold 120 is washed with deionized water and air-dried before plasma treatment. A plasma treatment system may be used, which includes a generator with a 40 kHz radio frequency signal. The mold 120 to be plasma activated is placed in the plasma chamber. The pressure is reduced to 0.1 mbar using a vacuum pump and the process gas (i.e., oxygen with 99% purity) is fed into the plasma chamber at the pressure chamber. When this operating pressure is reached, the generator is turned on and the process gas in the receiver is ionized. The plasma treatment duration varies between 10, 20, 30, 40, 50 and 60 minutes.

To form a microneedle patch 110, two layers of aqueous solutions are added to the microneedle mold 120 in a specific order. First, a mixture 150 of nucleic acids encapsulated by a plurality of lipid nanoparticles and polymeric solutions mixed in a 1:1 ratio is added to fill the array of recesses of the microneedle mold 120. After filling the recesses, a second solution 160 consisting solely of polymers is added to the microneedle mold 120 on top of the first layer of solution 150. Air gap between the two layers is eliminated by vacuuming. Pumped and released gas back three times in a vacuum chamber, each time lasting 3 mins, with an applied vacuum pressure of −1.0 bar. The microneedle mold 120 together with the two layers of solutions 150, 160 are then place in a blast drying oven for 24 h to dry at around 20° C., shaping the solutions into a microneedle patch 110 with a substrate and multiple needle-like features arranged on it. Due to the order in which the two solutions are added, only the microneedles contain nucleic acid drugs encapsulated within lipid nanoparticles, not the substrate. After cooling, the microneedles are removed from the mold 120.

The microneedle patch 110 is packed in a nitrogen (N₂) environment, preferably in a dry box and a moisture-proof box, and stored under room temperature (0-40° C.) and at a humidity of about 40% or less, without the direct sun light.

An array of needle-like features 170 of a microneedle patch 110, FITC-LNP-MN, is demonstrated in the fluorescence image of FIG. 1b, while FIG. 1c shows a photograph of such microneedle patch 110.

FIG. 1d also shows a photograph of another embodiment of the microneedle patch 110, mRNA-LNP.

The lipid nanoparticles containing nucleic acids are manufactured using microfluidics (Y- or T-type microfluidics), where water and ethanol are taken as the solvents of drugs and lipids respectively.

### 3. Synthesis of Lipid Nanoparticles and Encapsulation of Nucleic Acid Drugs

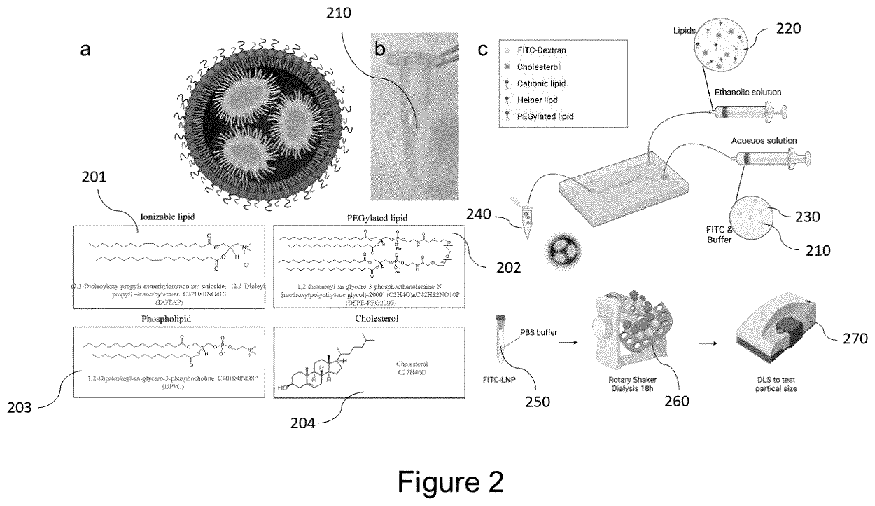

Lipid nanoparticles (LNPs) are formed using microfluidic technology to encapsulate mRNA and small molecule drugs, a cross section of a lipid nanoparticle is shown in FIG. 2a. In an embodiment, the synthesis of lipid nanoparticles comprises four components, including (i) DOTAP chloride 201, a highly efficient cationic lipid, (ii) Dipalmitoylphosphatidylcholine (DPPC) 203, a phospholipid, (iii) 1,2-distearoyl-sn-glycero-3-phosphoethanolamine-N-[methoxy-(polyethylene glycol)-2000] (DSPE-PEG(2000)) 202, and cholesterol 204, as shown in FIG. 2a.

FIG. 2c shows that the four components being dissolved in ethanol 220 in a molar ratio of 30:17.2:2.8:50. This mixture is then quickly combined with 52 μg/ml of EGFP-mRNA 240 dissolved in sodium citrate buffer (pH=4) 230, and 3 mg/ml of FITC-Dextran 210 (the model drug) dissolved in sodium citrate buffer 230 (pH=6). FIG. 2B displays a photograph of the FITC-Dextran solution 210.

Nanosuspensions are created by passing the mixture in organic and aqueous phases through a microfluidic chip at a flow rate of 1:3. The resulting mRNA-LNP and FITC-LNP 250 nanosuspensions are then dialyzed using a dialysis cup to remove ethanol. Afterwards, centrifuge tubes are filled with phosphate-buffered saline (PBS) and spun on a rotary shaker 260 for 18 hours in a dark environment, specifically for FITC-LNP. The PBS solution at the bottom is replaced every two hours. Finally, the dialyzed nanosuspensions were transferred to 2 ml centrifuge tubes.

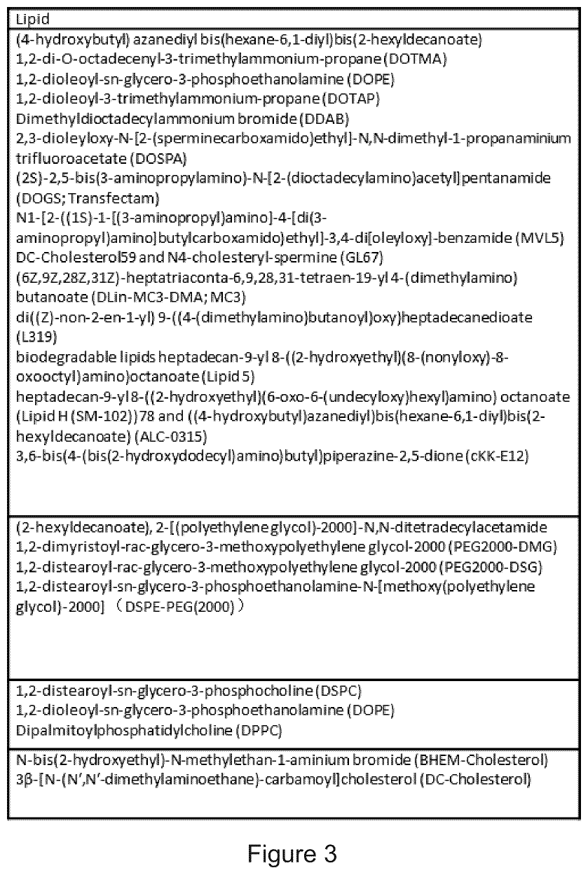

Alternatively, lipid nanoparticles may also be formed with components listed in FIG. 3, which include (4-hydroxybutyl) azanediyl bis(hexane-6,1-diyl)bis(2-hexyldecanoate), 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), 2,3-dioleyloxy-N-[2-(sperminecarboxamido)ethyl]-N,N-dimethyl-1-propanaminium trifluoroacetate (DOSPA), (2S)-2,5-bis(3-aminopropylamino)-N-[2-(dioctadecylamino)acetyl]pentanamide (DOGS; Transfectam), N1-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol59, N4-cholesteryl-spermine (GL67), (6Z,9Z,28Z,31Z)-heptatriaconta-6,9,28,31-tetraen-19-yl 4-(dimethylamino) butanoate (DLin-MC3-DMA; MC3), di((Z)-non-2-en-1-yl) 9-((4-(dimethylamino)butanoyl)oxy)heptadecanedioate (L319), biodegradable lipids heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102))78 and ((4-hydroxybutyl)azanediyl)bis(hexane-6,1-diyl)bis(2-hexyldecanoate) (ALC-0315), 3,6-bis(4-(bis(2-hydroxydodecyl)amino)butyl)piperazine-2,5-dione (cKK-E12), (2-hexyldecanoate), 2-[(polyethylene glycol)-2000]-N,N-ditetradecylacetamide, 1,2-dimyristoyl-rac-glycero-3-methoxypolyethylene glycol-2000 (PEG2000-DMG), 1,2-distearoyl-rac-glycero-3-methoxypolyethylene glycol-2000 (PEG2000-DSG), 1,2-distearoyl-sn-glycero-3-phosphoethanolamine-N-[methoxy(polyethylene glycol)-2000] (DSPE-PEG(2000)) 1,2-distearoyl-sn-glycero-3-phosphocholine (DSPC), 1,2-dioleoyl-sn-glycero-3-phosphoethanolamine (DOPE), Dipalmitoylphosphatidylcholine (DPPC N-bis(2-hydroxyethyl)-N-methylethan-1-aminium bromide (BHEM-Cholesterol), and 3β-[N-(N′,N′-dimethylaminoethane)-carbamoyl]cholesterol (DC-Cholesterol).

### 4. Optimization of LNP

Lipid nanoparticle (LNP) including liposomes, solid lipid nanoparticles, nanostructured lipid carriers, and polymer-lipid hybrid nanoparticles, is an emerging type of drug carriers. While the initial idea comes from liposome (with a lipid bilayer structure), it has evolved into more complex architectures with broad physicochemical characteristics. However, their roles in drug delivery are similar: to enhance the solubility, stability, and bioavailability; to reduce the toxicity and side effect. Their success has been documented by the United States Food and Drug Administration (FDA) approval of cancer drugs like Doxil and COVID-19 mRNA vaccines.

Transdermal delivery of these LNPs is useful for vaccination and treating local skin problems with less invasiveness and better patient compliance. Microneedle (MN) is one emerging strategy to realize the transdermal delivery of LNPs in a minimally invasive manner. In this concept, LNPs are either embedded in the dissolvable MN matrix or coated on the surface of non-dissolvable MNs. When MNs break through the stratum corneum and reach the epidermis and dermis layers, LNPs detach and diffuse across the skin to subcutaneous tissues and capillary vessels.

To develop an effective and efficient LNP-MN delivery system, researchers have to optimize the physicochemical properties of LNPs (i.e. size and charge) that regulate nano-bio interactions and to screen MN materials and fabrication procedure for sufficient mechanical properties of the ultimate device. Currently, the optimization is done through trial and error, heavily influenced by personal experience and preference of the researchers.

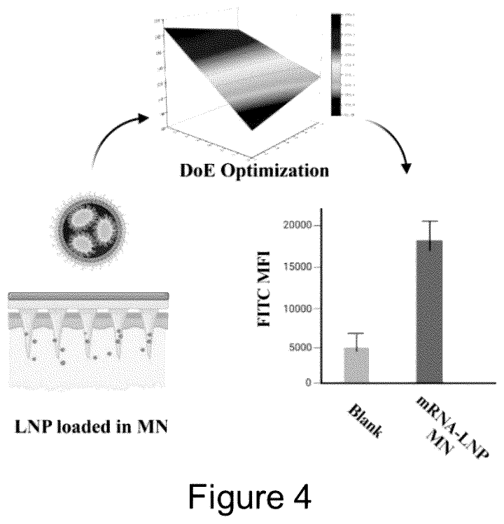

Design of Experiments (DOE) is a systematic, efficient method to study the relationship between multiple input and output variables, which is used in the development of the present invention for optimizing the key process parameters in the LNP-MN fabrication so as to gain independence from personal experience and preference. FIG. 4 demonstrates an optimising process of forming a microneedle patch, by utilising DOE. Specifically, the particle size of LNP and formulation of MNs are optimised through DOE. The resulted LNP-MN device can release 120 nm LNPs (PDI<0.2) upon the dissolution, which can transfect the fibroblasts successfully with both organic dye molecules and EGFP-mRNA.

### 4.1 Synthesis and Optimization of LNPs Using DOE Approach

LNPs, composing of four lipid components were synthesized using a Y-shape microfluidic device. These components include cationic lipids such as (2,3-Dioleoyloxy-propyl)-trimethylammonium-chloride and (2,3-Dioleyl-propyl)-trimethylamine (DOTAP), as well as 1,2-Dipalmitoyl-sn-glycero-3-phosphocholine (DPPC), 1,2-distearoyl-sn-glycero-3-phosphoethanolamine-N-[methoxy(polyethylene glycol)-2000] (DSPE-PEG 2000), and cholesterol.

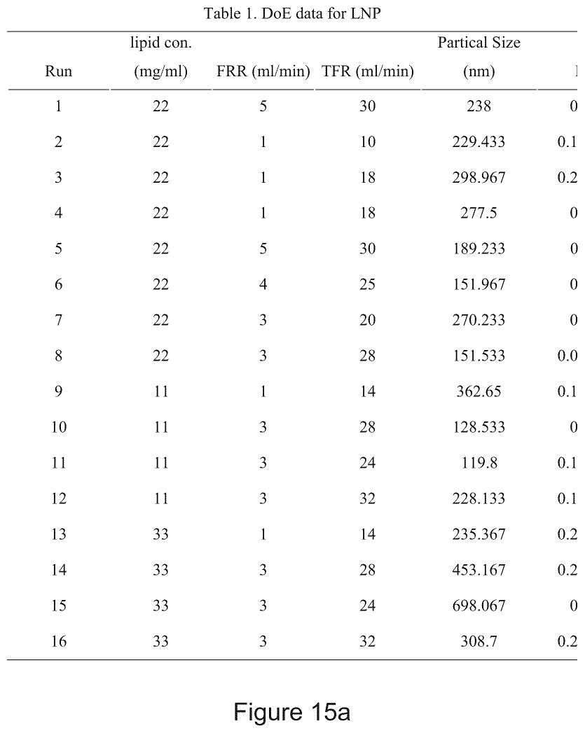

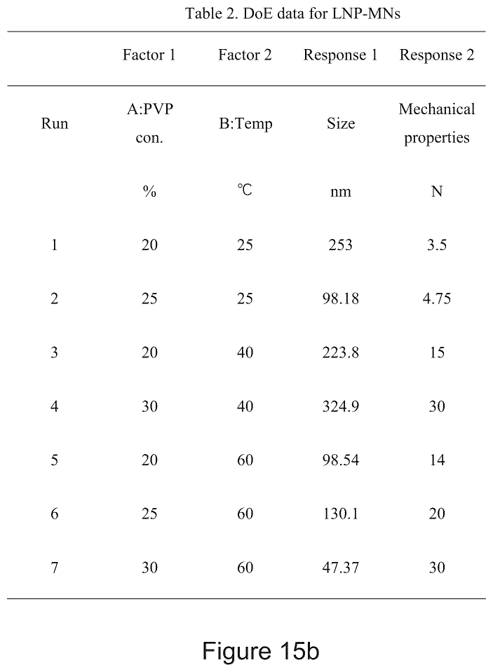

Fluorescein isothiocyanate-Dextran (FITC-Dextran, MW=250,000 Da) was used as the model drug for optimization. The LNP size was adjusted to be between 125 and 190.4 nm (PDI ranging from 0.073 to 0.45) by varying the total lipids' concentration and the total flow rate (TFR) between the aqueous and organic phases using a 2² full factorial design, as referred to the table of FIG. 15a.

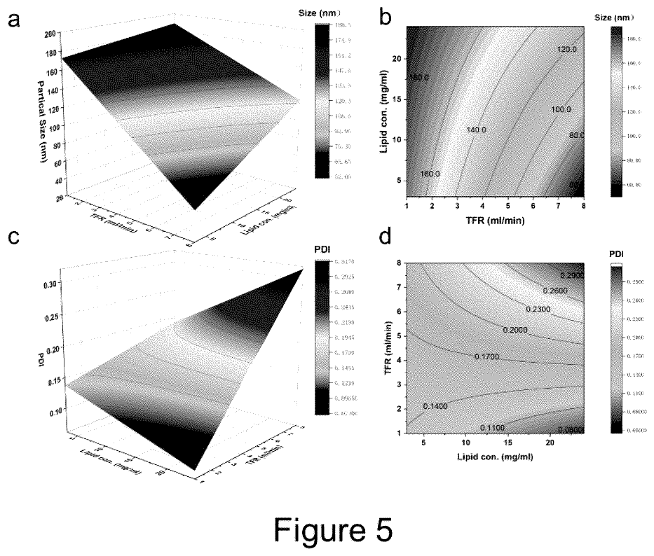

During the experiment, the flow rate ratio (FRR) was fixed at 3, while the molar ratio between DOTAP, DPPC, DSPE-PEG 2000, and cholesterol was fixed at 30:18:2:50. The output variables were LNP size and PDI, while the input variables were lipid concentration and TFR. The correlation between the factors and the responses can be observed visually in the three-dimensional (3D) plot of DOE, as shown in FIGS. 5a and 5c.

The optimal size for LNPs is 120±30 nm with a PDI of less than 0.2 to ensure efficient cellular internalization. FIG. 5a shows a response surface plot of DOE for the optimization of LNP sizes with TFR versus lipid concentration. The predicted R² value of 0.8891 is in reasonable agreement with the adjusted R² value of 0.9668 (the difference is less than 0.2). R² is a statistic that measures the goodness of fit of a regression model to the observed data. It is a value between 0 and 1, with a higher value indicating a better fit of the model to the data. FIG. 5a shows that the LNP size decreases as the TFR increases, regardless of the total lipid concentration value.

However, the changes in LNP size due to TFR variations are more significant when the total lipid concentration is low. FIG. 5b shows the contour lines indicating that the LNP size is approximately 120±30 nm when the TFR and lipid concentration are in the range of 8-4 ml/min and 24-3 mg/ml.

Additionally, PDI is examined under different synthetic conditions (P-value<0.0001). A PDI of less than 0.2 in DLS measurement indicates a uniform size distribution of LNPs. FIG. 5c demonstrates that the PDI increases with an increase in TFR, regardless of the lipid concentration. The variation of PDI due to the changes of TFR is more significant when total lipid concentration is larger.

The correlation between lipid concentration and PDI has two stages, as shown in FIG. 5c. At lower flow rates (TFR<4.00 ml/min), the PDI decreases with an increase in lipid concentration. This may be because a higher concentration of lipids leads to better control of lipid self-assembly. When TFR is between 4-8 ml/min, higher lipid concentrations result in a larger PDI. This is because higher lipid concentrations may lead to a faster self-assembly process, resulting in a wider range of particle sizes as not all particles have sufficient time to grow to the same size.

The contour plot in FIG. 5d demonstrates that PDI is larger than 2.0 only when TFR and lipid concentration are not distributed simultaneously within the ranges of 8-5 ml/min and 24-5 mg/ml. To ensure better control of the production process, a TFR between 6-1 ml/min and a total lipid concentration between 3-14 mg/ml is chosen.

In summary, an optimal LNP formulation should have a narrow particle size distribution of 120±30 nm (PDI<0.2). This can be achieved by using a TFR of 4-6 ml/min and a lipid concentration range of 3-14 mg/ml.

4.2 Synthesis and Characterization of FITC-Dextran-LNPs or mRNA-LNPs

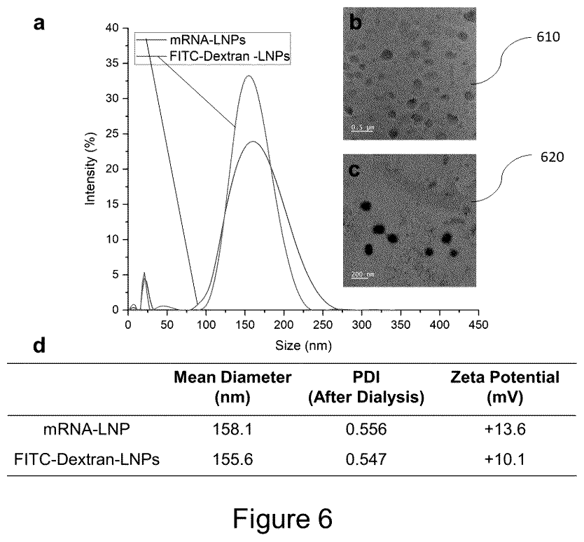

FITC-Dextran-LNPs and mRNA-LNPs were prepared using an optimized total lipid concentration of 6.5 mg/ml and TFR of 5.2 ml/min. Both LNPs had hydrodynamic diameters of approximately 140 nm (PDI, 0.18), which increased slightly after dialysis to around 150 nm. The zeta potentials of the LNPs were +10.1 mV and +13.6 mV, respectively, as shown in FIGS. 6a and 6d.

FIGS. 6b and 6c are Transmission Electron Microscopy (TEM) images of mRNA-LNPs and FITC-Dextran-LNPs, which show that both FITC-Dextran-LNPs 620 and mRNA-LNPs 610 have a relatively homogeneous shape with an external bilayer and an electron-dense internal amorphous structure. The percentage of drugs in the LNPs was also quantified. 1 mg of FITC-Dextran-LNPs contained 0.3775 mg of FITC-Dextran, while 1 mg of mRNA-LNPs contained 0.02374 mg of mRNA.

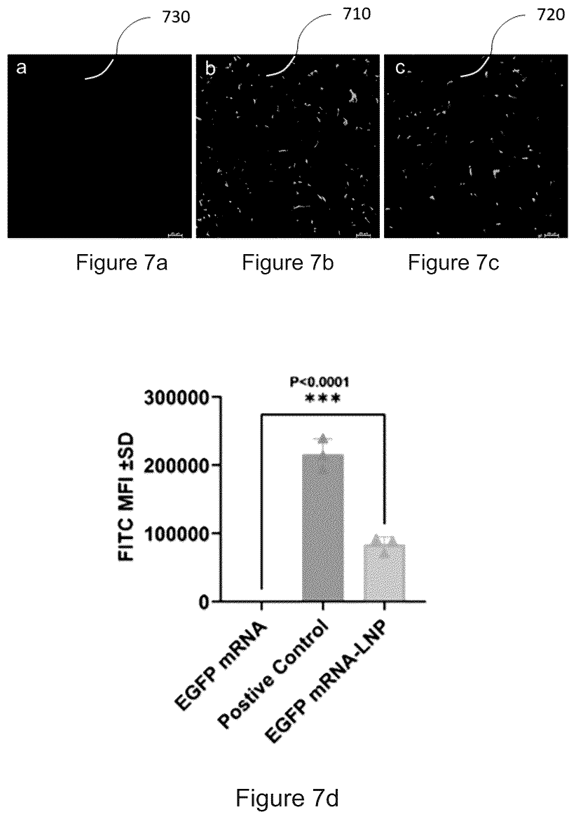

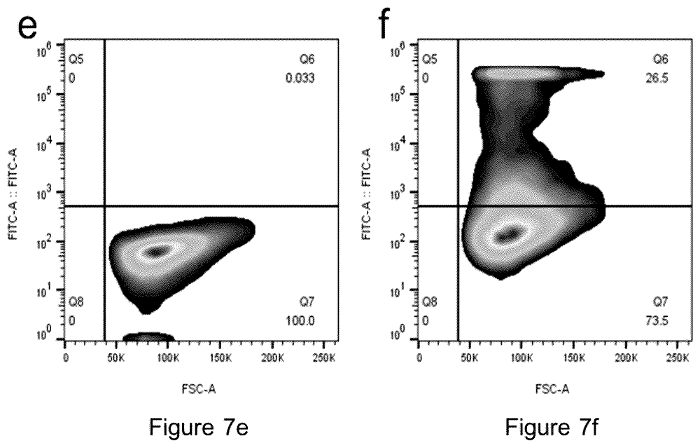

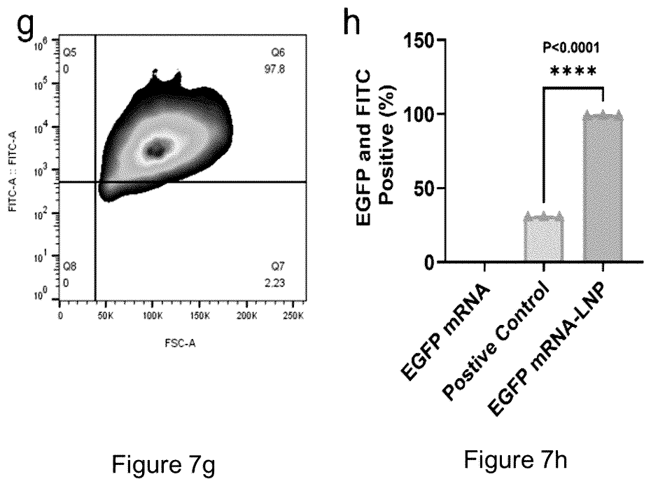

These LNPs were used to transfect mesenchymal stem cells (MSCs). The experiment included negative controls (FITC-dextran or mRNA alone), positive controls (molecules transfected using commercial Lipofectamine), and LNPs. The results of EGFP mRNA transfection were examined through fluorescence imaging, as shown in FIGS. 7a to 7c. Both Lipofectamine 710 and LNP 720 transfected cells exhibited fluorescence. Specifically, cells transfected with Lipofectamine show stronger average fluorescence per fluorescent cell, as shown in FIG. 7d.

In contrast, negative control cells 730 showed no detectable EGFP fluorescence. These cells were further examined through flow cytometry analysis, as shown in FIGS. 7e to 7g. Interestingly, in the mRNA-LNP group, there was 99.4% EGFP positive cells while the number was only 31.0% in the lipofectamine group illustrated by FIG. 7h.

For FIGS. 7d-7h, values are expressed as mean±SD. Error bars indicate SD values from three independent experiments with three donors. Statistical differences are expressed as **p<0.005, ***p<0.001.

The difference between the fluorescence quantification and flow analysis suggests that LNPs provided a more homogenous transfection while lipofectamine could bring more molecules into some portions of cells. This can be confirmed through the flow data of FIGS. 7e to 7h. In FIG. 7f which refers to the lipofectamine group, the distribution of cells with different fluorescence intensity is much broader than that in FIG. 7g which refers to the LNP group.

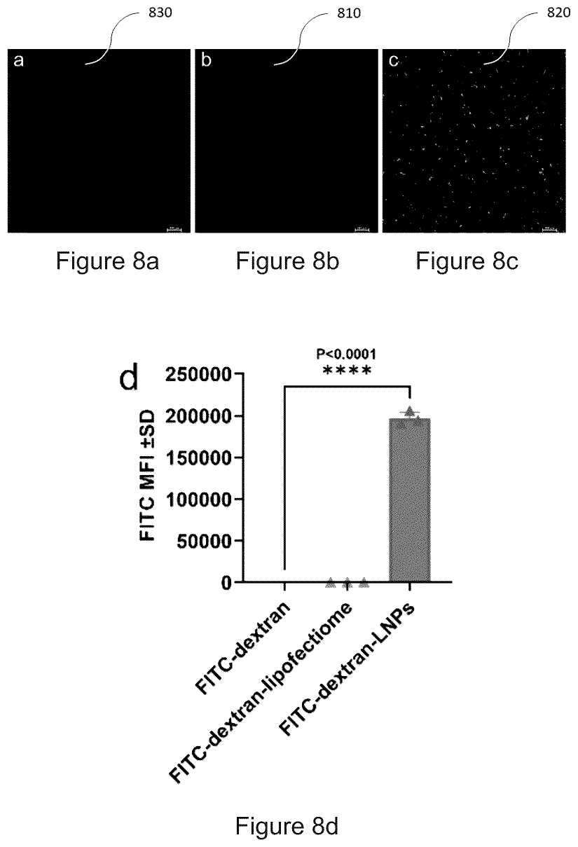

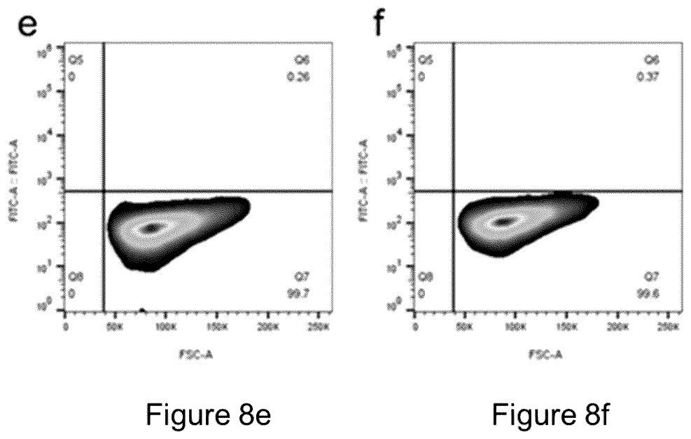

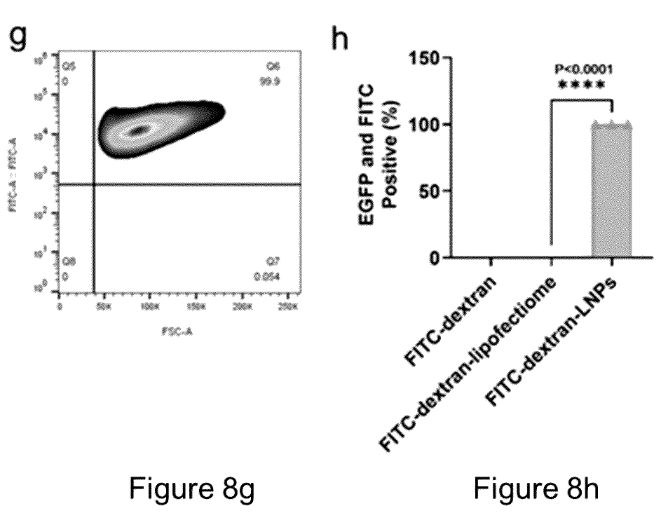

Referring now to FIG. 8, when FITC-dextran was the molecules to be delivered, lipofectamine and LNPs showed totally different results. In the fluorescence images of FIGS. 8a to 8c, and the flow cytometry analysis of FIGS. 8e to 8g, lipofectamine 810 did not bring FITC-dextran into the cells. In contrast, LNPs 820 successfully transfected cells with FITC-dextran.

Similar to that in the mRNA transfection, the transfection as shown in FIG. 8g was homogenous as well. The failure of FITC-dextran transfection using lipofectamine 810 can be explained by the zeta potential of the FITC-dextran-lipofectamine conjugate. In mRNA transfection, the EGFP-mRNA-lipofectamine 820 showed a zeta potential of +6.310 mV in DMEM, which would allow the complexation between cell membrane and EGFP-mRNA-lipofectamine conjugate. However, FITC-dextran-lipofectamine conjugates 810 showed a negative zeta ζ potential in DMEM, which would not facilitate the complexation.

For FIGS. 8d-8h, values are expressed as mean±SD. Error bars indicate SD values from three independent experiments with three donors. Statistical differences are expressed as **p<0.005, ***p<0.001.

Fibroblast and HEK 293 are successfully transfected using FITC-dextran-LNPs. As shown in FIGS. 14a-14c which show the fluorescence images of fibroblast cells transfected with a) FITC-dextran alone, b) FITC-dextran-lipofectamine and c) FITC-dextran-LNPs, lipofectamine 1410 did not bring FITC-dextran into fibroblast cells as no fluorescence is shown in FIG. 14b. In contrast, LNPs 1420 successfully transfected fibroblast cells with FITC-dextran.

FIGS. 14d-14f show the fluorescence images of HEK 293 cells transfected with a) FITC-dextran alone, b) FITC-dextran-lipofectamine and c) FITC-dextran-LNPs. Similarly, Lipofectamine 1430 did not bring FITC-dextran into HEK 293 cells as no fluorescence is shown in FIG. 14e, while LNPs 1440 successfully transfected HEK 293 cells with FITC-dextran.

### 4.3 Fabrication and Optimization of Dissolvable LNP-MN Formulations Using DOE Approach

LNP-MNs were fabricated by the template melding method, where the solution containing polymer and mRNA-LNP or FITC-dextran LNPs was loaded into the negative MN mold and then dried. The concentration of polymer (PVP) and drying temperature were key parameters in this process.

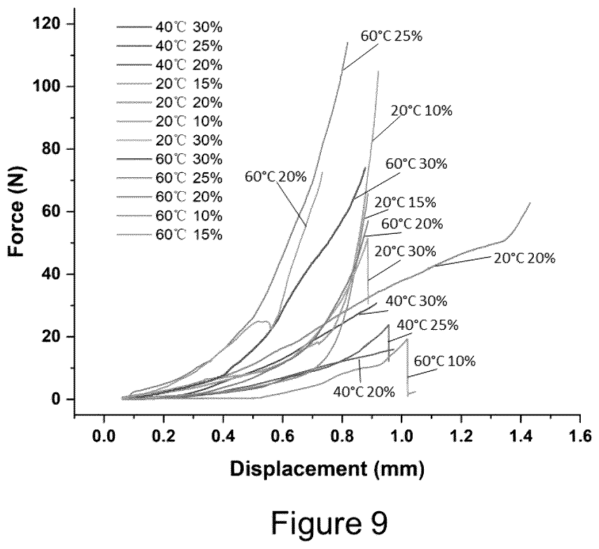

The Young's modulus of soluble MN was tested using a tensile tester. FIG. 9 shows the relationship between the load force/patch and the tip displacement of LNP-MN with different concentrations (30%, 25%, 20%, 15%, 10%) of PVP under drying conditions at 20° C., 40° C. and 60° C.

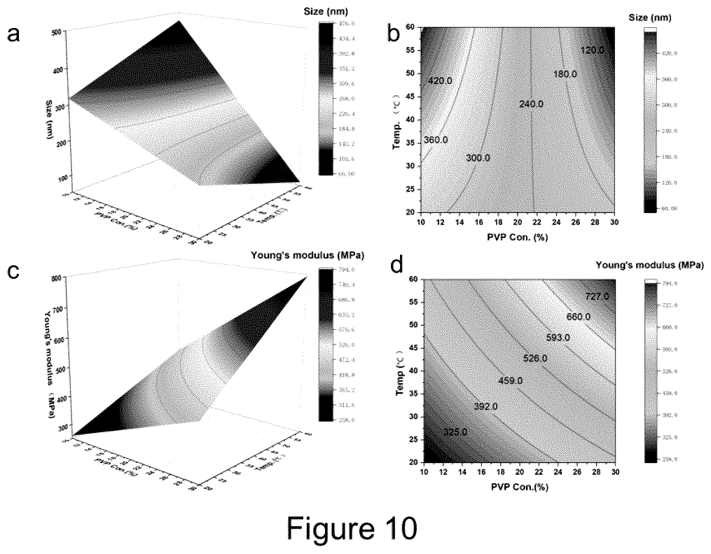

The output variables, as known as responses, are the Young's modulus of LNP-MNs and the sizes of LNPs following the dissolution of LNP-MNs. The Young's modulus was derived from the force-displacement curve of compression test, as shown in FIG. 9. Young's modulus is a mechanical property of solid materials that measures the tensile or compressive stiffness when the force is applied lengthwise. The correlation between the factors and the responses was displayed in the 3D plot of DOE, as illustrated in FIG. 10. The concentration of PVP was adjusted between 10-30%, and the drying temperature was within the range of 20° C. to 60° C.

The ideal LNP-MNs should have a high Young's modulus and LNPs released from LNP-MNs have the same hydrodynamic diameter as the unencapsulated LNPs (150 nm). Across all the drying temperatures, a lower PVP concentration is beneficial to keep the particle sizes, as shown in FIG. 10a. This should be due to the high viscosity of solution at higher PVP concentration promoted the aggregation of the nanoparticles.

FIG. 10a shows the response surface plots of the size of LNP released at different PVP concentrations (10-30%) and drying temperatures (20° C., 40° C., 60° C.). Ideally, LNP-MNs should have a high Young's modulus, and the LNP released from LNP-MNs has the same hydrodynamic diameter (150 nm) as the unencapsulated LNP. Higher PVP concentrations are favourable for maintaining particle size at all drying temperatures. This is supposed to be due to the fact that the higher the PVP concentration, the higher the viscosity of the solution, which prevents the aggregation of the nanoparticles. From the contours (FIG. 10b), it can be seen that the size of dissolved LNP is less than 180 nm at PVP concentrations and drying temperatures of 24-30% and 22-60° C., respectively.

In addition to the dispersion of LNP-MNP, the described study also examined the effect of different manufacturing conditions on the Young's modulus properties of the microneedle materials. As shown in FIGS. 10c and 10d, the mechanical properties of MNP were clearly affected by PVP concentration and drying temperature. The Young's modulus of the microneedles increased with the increase of drying temperature when the PVP concentration was between 10% and 30%. The mechanical properties of microneedles increased with increasing PVP concentration at temperatures between 20° C. and 60° C. The mechanical properties of microneedles increased with increasing PVP concentration. Since mRNA is easily degraded at high temperatures, we tried to dry mRNA-LNP at lower temperatures. therefore, we tried to obtain LNP-MNP with higher Young's modulus at lower drying temperatures.

The force per microneedle patch (100 tips) could be as high as 190,000 N/m2 at a 30% PVP concentration and a temperature of 25° C. The compressive strength of soluble MNP loaded with PVP for LNP was 19 MPa. which exceeds the force required for normal skin penetration (0.058 N). In addition, a concentration of 30% PVP and a drying temperature of 25° C. satisfy the requirement that the size of dissolved LNP be less than 180 nm.

4.4 Cell Transfection In Vitro by LNPs Released from LNP-MN

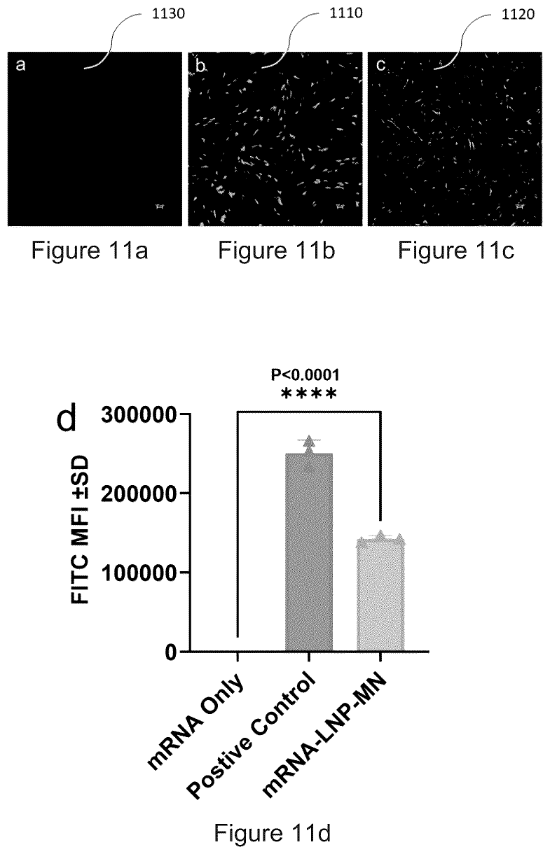

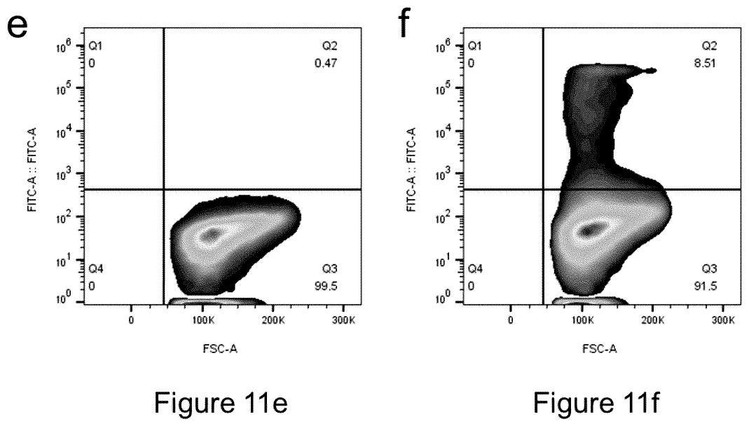

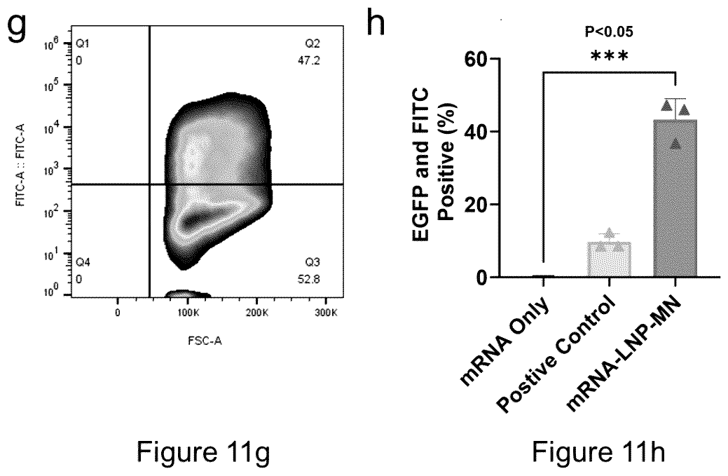

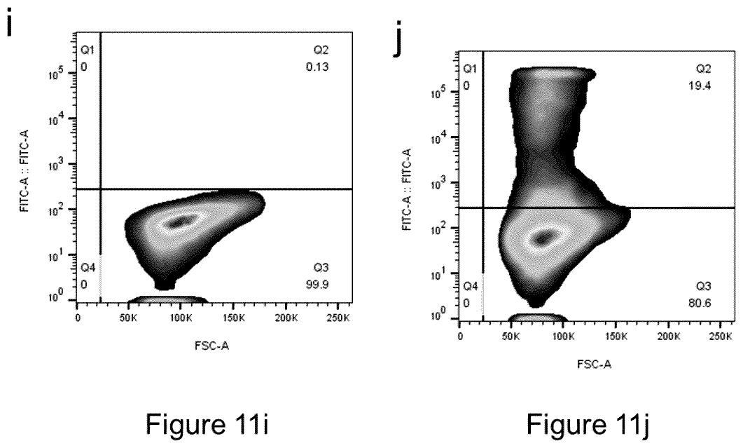

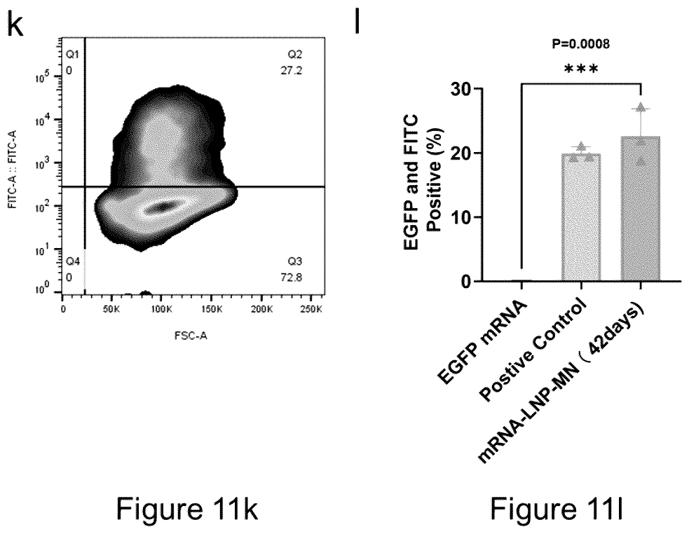

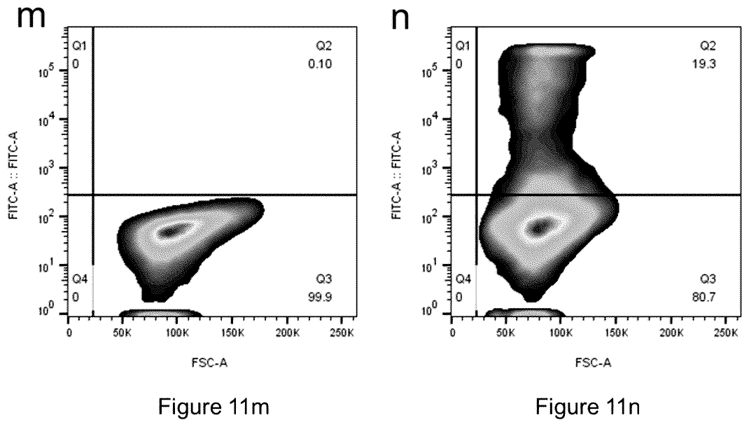

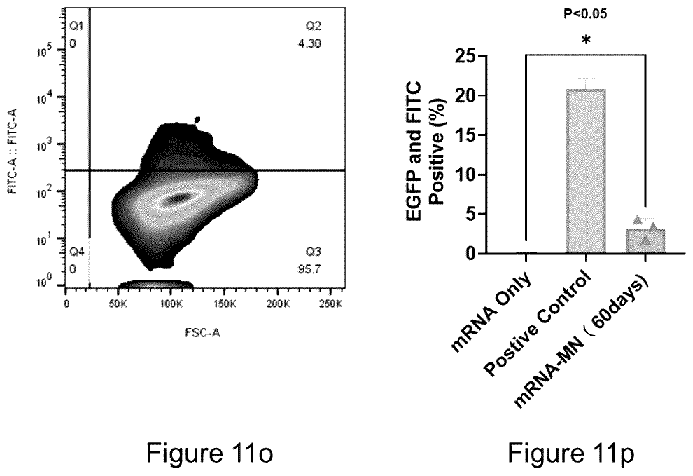

The optimized LNP-MNs were used to transfect mesenchymal stem cells (MSCs). Negative controls (FITC-dextran or mRNA only), positive controls (using commercial Lipofectamine) were also included. The results of EGFP mRNA-LNP-MN transfection were examined by fluorescence imaging, as shown in FIGS. 11a to 11c.

Cells transfected with both Lipofectamine 1110 and LNP-MNs 1120 exhibited fluorescence. The fluorescence intensity of the LNP-MNs 1120 was comparable to that of Lipofectamine 1110 transfected. This suggests that the microneedle fabrication and drying process did not damage the mRNA-LNPs.

Flow cytometry analysis (FIG. 11e-h) showed that the transfection efficiency of LNP-MN was more than 40%, which was much higher than that of the positive control group (8.51%), and was more homogeneous than that of lipid copy amine.

In contrast, the negative control cells did not exhibit detectable EGFP fluorescence, as shown in FIG. 11d.

These cells were further examined by flow cytometry analysis, as illustrated in FIGS. 11e to 11g. Similar results of the LNPs transfected group were obtained. It is demonstrated that LNP-MNs provide a more homogeneous transfection. As shown in FIGS. 11i-11l, the manufactured mRNA-LNP-MN patches were stored in a sealed container for 42 days and then transfected into MSC cells, wherein the storage temperature was set at 23° C., with a humidity range of 50%-63%.

In particular, FIG. 11h shows the transfection of the mRNA-LNP-MN group used directly after production, with a mean value of 43.3% for egfp positive (%). This is in comparison to the transfection result of the same group after 42 days, which shows a mean value of 22.633% for eGFP-positive (%). As shown by the results, mRNA-LNP-MNs still maintained more than 50% of the original transfection efficiency after 42 days.

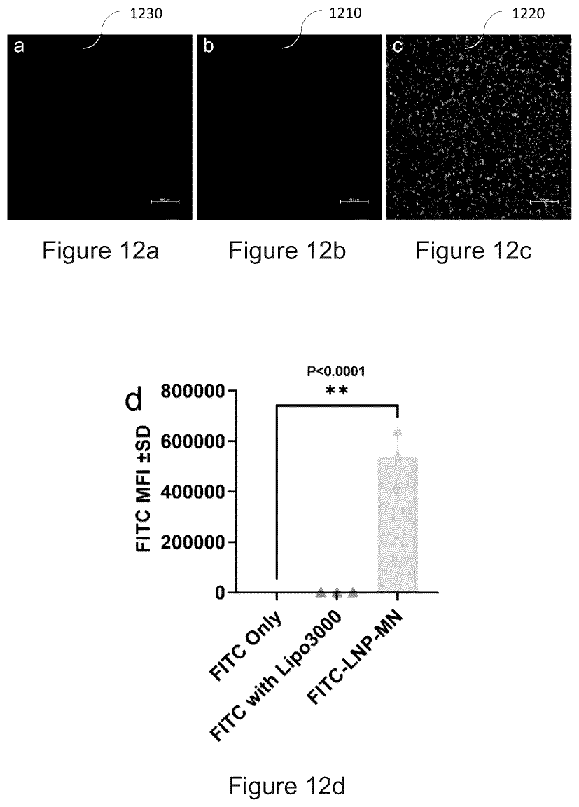

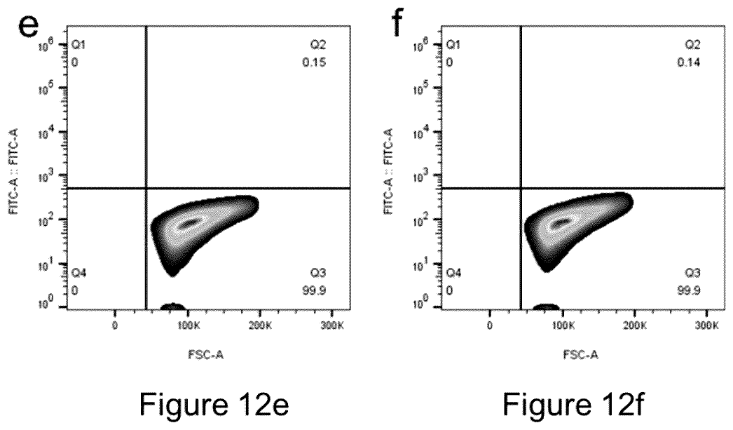

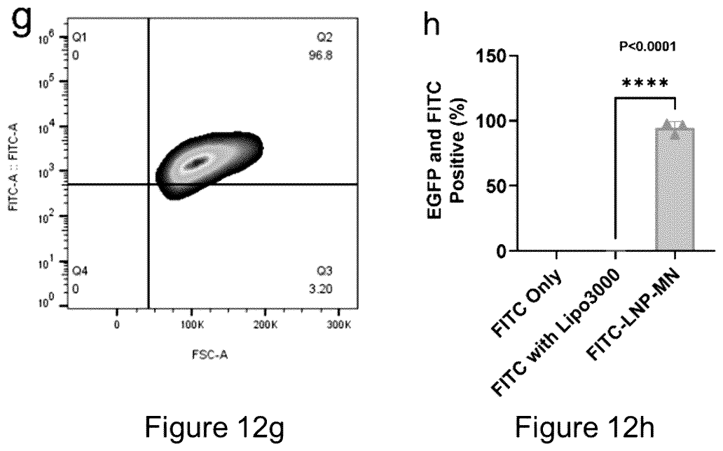

FIGS. 11m-11o illustrate the transfection results of FIGS. 11e-11g after 60 days of storage; wherein FIG. 11p shows the quantification of fluorescence intensity of cells in the flow cytometry analysis of FIGS. 11m-11o. As illustrated in FIG. 11p in particular, the transfection of the mRNA-LNP-MN group after 60 days of storage exhibited a mean value of 3.14% for eGFP-positive cells. The transfection result after 60 days remained significantly higher than that of mRNA only, thereby demonstrating the biological activity of the drug after 60 days. Referring to FIG. 12, when delivering FITC-dextran via LNP-MNs, only LNP-MNs 1220 brought FITC-dextran into the cells, but not lipofectamine 1210 as shown in fluorescence images of FIG. 12a to 12c, and flow cytometry analysis of FIGS. 12e to 12g. The transfection efficiency of FITC-dextran-LNP-MN was very high, which was more than 95%, which proved that LNP in soluble microneedles could be well absorbed by cells.

For FIGS. 11d-11h and FIGS. 12d-12h, values are expressed as mean±SD. Error bars indicate SD values from three independent experiments with three donors. Statistical differences are expressed as **p<0.005, ***p<0.001.

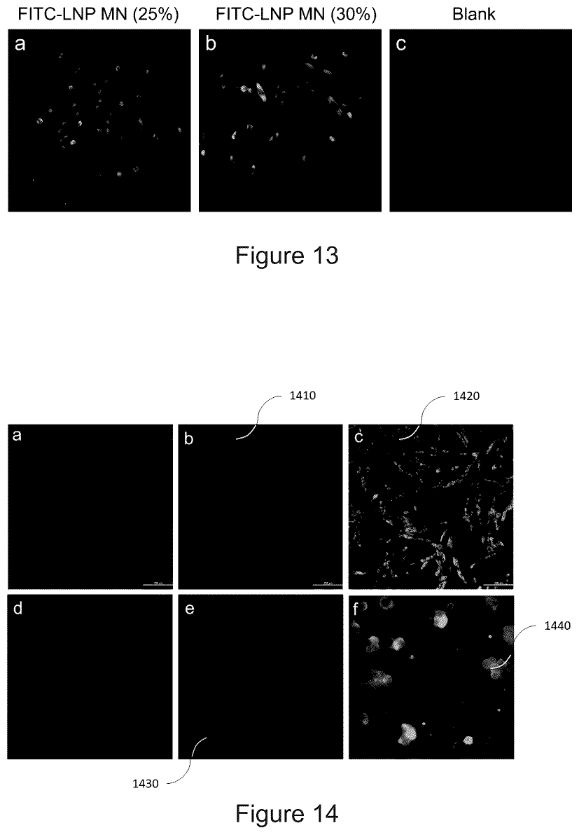

The fibroblasts were also successfully transcribed using FITC-dextran-LNP-MN fabricated with a high drying temperature of 60° C., as shown in FIG. 13.

FIG. 13a shows the fluorescence image of fibroblast cells transfected with FITC-dextran-LNP-MN fabricated by 30% of PVP after drying at 60° C.; FIG. 13b shows the fluorescence image of fibroblast cells transfected with FITC-dextran-LNP-MN fabricated by 30% of PVP after drying at 60° C.; while FIG. 13c shows the fluorescence image of fibroblast cells transfected with FITC-dextran alone.

It can be seen that florescence is present in both FIGS. 13a and 13b, showing that LNPs are stabile during the drying process of MNs.

This suggests that the dissolvable LNP-MN delivery system has great potential for delivering nucleic acids, proteins, and small molecules with DOE optimization process. Process parameters for LNP-MNP can be designed based on the structure, physicochemical properties, and required storage conditions of the drugs to optimize the efficiency of microneedle vaccine delivery.

### 5. Discussion

In this invention, an advanced soluble LNP-MN patch is constructed. Through the DOE method, problems of long test periods and high test cost are avoided, as well as the rough test methods, and the inability to effectively assess the interactions between inputs. It is significant in the large-scale design and manufacture of MN to meet the market demand for personalized medicine.

The influencing factors in the microfluidic fabrication of LNPs and the relationship between their interactions are also being investigated. In the large-scale synthesis of LNPs, the flow rate is controlled as much as possible; otherwise, too high a flow rate will not only fail to reduce the particle size of the LNP, but will also make it difficult to achieve continuous and stable LNP synthesis, resulting in excessive batch-to-batch error.

Meanwhile, the study on the manufacturing of microneedles is also aimed at optimizing its mechanical and dispersion properties. In addition to this, it is also possible to compare the effects of different materials such as PVA, sucrose, maltose, etc. on the performance of microneedles, and to consider the preservation and transportation conditions, as well as high-temperature manufacturing of nucleic acids and other drugs in the damage.

### 6. Materials and Methods

### 6.1 Synthesis of Lipid Nanoparticles

LNPs were prepared by microfluidic technology, which can be used to encapsulate mRNA and small molecule drugs. The microfluidic equipment was used to select DOTAP chloride a highly efficient cationic lipid, Dipalmitoylphosphatidylcholine (DPPC) a phospholipid, 1,2-distearoyl-sn-glycero-3-phosphoethanolamine-N-[methoxy-(polyethylene glycol)-2000] (DSPE-PEG(2000)), and cholesterol to synthesize lipid nanoparticles to synthesize lipid nanoparticles.

The four materials were dissolved in ethanol in a molar ratio of 30:17.2:2.8:50, rapidly combined with sodium citrate buffer (pH=4) dissolving 52 μg/ml of egfp-mRNA (Dakewe) and sodium citrate buffer (pH=6) dissolving 3 mg/ml of FITC-Dextran (Sigma-Aldrich) respectively. Nanosuspensions were obtained by passing through the microfluidic chip at a flow rate 1:3 between the organic and aqueous phases. The obtained mRNA-LNP and FITC-LNP nanosuspensions were dialyzed out of ethanol in a dialysis cup Centrifuge tubes were filled with phosphate-buffered saline and spun on a rotary shaker for 18 hours, taking care to avoid light. The PBS solution at the bottom was replaced every two hours, and finally, the dialyzed Nanosuspensions were transferred to 2 ml centrifuge tubes.

### 6.2 Characterization of Physicochemical Properties of LNP

The microfluidically synthesized sample liposomes were characterized. LNP was analysed by dynamic light scattering (DLS). The particle size was measured by DLS and the PDI of LNP was analysed and diluted with phosphate-buffered saline (PBS) and added to a high-concentration cell equipped with a transparent electrode to measure the zeta potential. Three measurements per sample were averaged.

### 6.3 Transmission Electron Microscopy (TEM)

TEM photography of mRNA-LNP and FITC-dextran-LNP. Lipid nanoparticles were loaded onto specialised copper plates and then imaged using transmission electron microscopy. TEM provided high-resolution images revealing the morphology and diameter of LNP.

### 6.4 LNP Encapsulation Efficiency

The RNA assay kit was used to determine the percentages of encapsulated RNA and RNA concentrations, as previously described. To quantify the unencapsulated amount of RNA in the LNPs, the samples were diluted to an expected total RNA concentration of 10 μg/mL in 1× TE buffer before addition of the RiboGreen dye. To quantify the total amount of RNA, the samples were diluted to an expected total RNA concentration of 10 μg/mL in 1× TE buffer containing 0.5% (v/v) Triton X-100 before addition of the dye. Standard solutions were prepared in 1× TE containing 0.5% (v/v) Triton X-100 at final RNA concentrations of 0-2.5 μg/mL. The assay was carried out according to the manufacturer's protocol. Fluorescence intensities were measured at an excitation of 480 nm and emission of 520 nm. Dosing for transfection was based on the calculated encapsulated dose.

$$EE \% = \frac{mass of encapsulated RNA}{total mass of RNA used} \times 100 \%$$

For FITC-LNP via a similar method as above, since FITC-Dextran fluorescence intensity varies with its concentration. A standard curve of fluorescence intensity versus FITC-Dextran concentration was plotted by dissolving different concentrations of FITC-Dextran with PBS buffer. The plate was read by a Microplate Reader to obtain the FITC-LNP fluorescence intensity in the dialyzed sample, and the concentration could be obtained by calculation.

### 6.5 Optimization of LNP

A three-factor two-level 24 full factorial design was employed to examine the main effects and interactions of different processing and formulation parameters. and to determine the optimum formulation parameters of FITC-LNP. The factors examined were A: total flowrate (which affects Re number) B: DOTAP concentration C: DOTAP-DSPE-PEG(2000) ratio (w/w) Response factors (i.e., critical mass attributes were: particle size, polydispersity index (PDI) and zeta potential. The levels of each variable are denoted as −1, 0, and +1. The exact test conditions are listed in Table 1. The experiments were conducted 18 times with a center point of 2×23+2 times.

6.6 Loading of mRNA-LNP and FITC-LNP into MNs

### Fabrication of Dissolved Microneedles

MN molds were prepared first, and each was subjected to Plasma treatment (30S) to enhance its hydrophilicity. Oxygen plasma treatment was washed with deionized water and air-dried before plasma treatment. A plasma treatment system was used in the study, which used a generator with a 40 kHz radio frequency signal. The molded samples to be plasma activated were placed in the plasma chamber. The pressure was reduced to 0.1 mbar using a vacuum pump and the process gas (i.e., oxygen with 99% purity) was fed into the plasma chamber at this pressure. chamber. When this operating pressure is reached, the generator is turned on and the process gas in the receiver is ionized. The plasma treatment time varied between 10, 20, 30, 40, 50 and 60 minutes.

The mRNA-LNP & FITC-LNP and polymer (PVP) solutions were mixed in a 1:1 ratio and poured into the previously prepared MN mold. The air gap was eliminated by vacuuming. Afterwards, the molds were dried in an oven and the MNs were removed after cooling.

### 6.7 Optimization of Soluble Microneedle Performance

The performance of microneedles was also optimized by the DOE design of experiments. The factors studied were A: PVP concentration B: Oven drying temperature Response factors (i.e., critical mass attributes): mechanical properties of microneedles, and polydispersity index (PDI) of LNP after dissolution. The levels of each variable are denoted as −1, 0, and +1. The exact test conditions are listed in Table 1. The experiments were conducted 9 times with a center point of 2×2²+1 where PDI is a reference value only, to observe the dispersion of LNP after microneedle dissolution and compare the different formulations' storage effect. For example, glucose, sucrose, mannitol, etc. However, due to the fact that saccharides are susceptible to moisture, they are not easy to store and transport.

6.8 Transfection Experiments with mRNA-LNP and FITC-LNP

To test whether different cells could take up FITC-LNP. HEK-293 cells, MSCs, and Fibroblast cells were cultured at 37° C. in a humidified atmosphere of 5% CO₂ in DMEM medium with 10% FBS for HEK-293K and Fibroblast. Cells were inoculated into 6-well plates (30 w/well) and 350 ul of dialyzed FITC-LNP nanosuspension was added. A control group was also set up 4 ug/ml of mRNA and 3 mg/ml of FITC-Dextran was added to the cells. Positive control group: 6 ul of Lipofectamine 3000 combined with 4 ug/ml of EGFP-mRNA, mixing.

Each well was diluted with DMEM with 1% FBS to 1.5 ml/well and incubated in an incubator for 8 h. After transfection, the mixture was removed, withdrawn, and washed with PBS buffer 3 times. Replace the DMEM with 10% FBS incubate for 24-48 hours.

### 6.9 Endocytosis Test

The steps were the same as for regular transfection, and control experiments were set up at the time of transfection. The first group of control HEK293 cells was transfected according to the above procedure, and the second group was transfected by placing them in a refrigerator at 4° C. for 40 minutes, then taking them out, washing them with PBS and replacing them with DMEM 1% FBS culture medium without FITC-LNP and incubating them at 37° C. for 10 minutes. After removing the culture medium, replace it with DMEM (1% FBS) mixed with 500 ul and 250 ul of FITC-LNP and return to 4° C. and remove after 40 min. At the same time, take out the first group of cells, wash the two groups of cells three times with PBS, and replace the DMEM (10% FBS) with DMEM to observe the fluorescence intensity of the two groups of cells under fluorescence microscope and analyse the endocytosis of LNP by the cells.

### 6.10 Soluble Microneedle Transfection Test.

The prepared MNP loaded with FITC-Dextran-LNP was removed and photographed under a fluorescence microscope to observe whether FITC-Dextran-LNPs was present at the bottom of the needle tip. Dissolve MNP in DMEM (1% FBS) and transfect MSC for 4-8 hours. Then, DMEM was switched to 10% FBS and the cells were expressed for 48 hours. The fluorescence intensity was observed under a fluorescence microscope.

### 6.11 Evaluation of Transfection Efficiency by Fluorescent Microscopy

To measure EGFP expression in adherent MSC, cells were imaged 48 hours after transfection using a Nikon fluorescence microscope with an excitation light source of 488 nm. Image-J was used to analyse the fluorescence intensity of the microscopic images.

### 6.12 Measurement of Cell Transfection Efficiency by Flow Cytometry

Cells were collected after 48 hours of expression following transfection for further washing with PBS and analysed by flow cytometry. Transfected MSC cells were collected by pipetting the collected cells after a washing step using flow cytometry washing solution. All flow cytometry data were analysed using FlowJo software V10.

### 6.13 Statistical Analysis

All data were analysed with GraphPad Prism version 9.0 (GraphPad Software Inc.) and expressed as Mean±standard error of the mean (SEM). The difference between groups was tested using either one-way or two-way analysis of variance (ANOVA). Specific statistical analysis methods are described in the figure legends where results are presented. Values were considered statistically significant for p<0.05.

### 7. Further Information

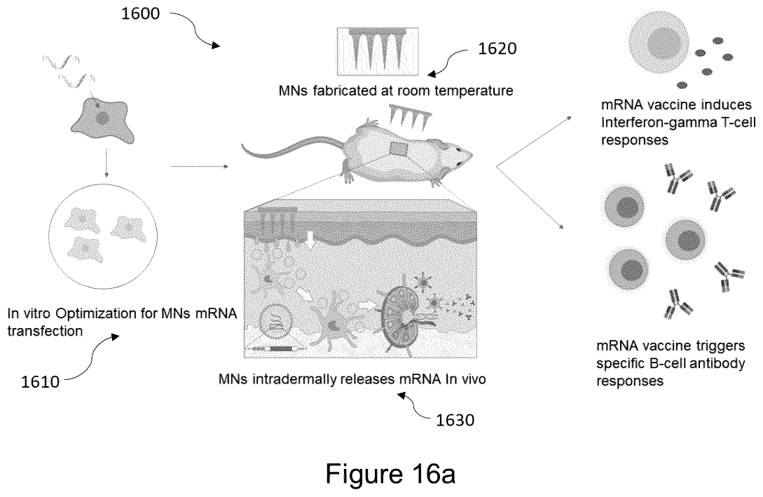

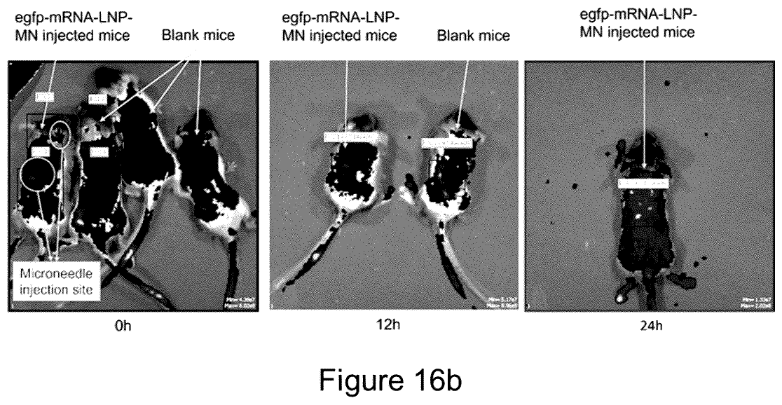

FIG. 16a demonstrates the intradermal delivery process 1600 of mRNA to mice using an embodiment of a microneedle patch according to the present invention. Upon optimisation in vitro 1610 for mRNA transfection is performed, an ideal microneedle patch is formed. Such microneedle patch is fabricated and applied to the mice at room temperature (0-40° C.) 1620. When the microneedle patch is applied to the skin of the mice, the microneedles intradermally release mRNA in vivo 1630. Results of the intradermal delivery process 1600 of mRNA to mice at 0 h, 12 h and 24 h are referenced in FIG. 16b.

The exemplary embodiments are thus fully described. Although the description referred to particular embodiments, it will be clear to one skilled in the art that the invention may be practiced with variation of these specific details. Hence this invention should not be construed as limited to the embodiments set forth herein.

While the embodiments have been illustrated and described in detail in the foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

1. A microneedle patch comprising a substrate and a plurality of microneedles disposed on the substrate, wherein each of the plurality of microneedles comprises:
(i) one or more polymers dissolvable in human skin layer,
(ii) a plurality of lipid nanoparticles, and
(iii) a nucleic acid drug encapsulated by the plurality of lipid nanoparticles;
wherein each of the plurality of microneedles is defined with a tip end portion that enables dermal penetration, and wherein the microneedle patch is adapted to be stored at room temperature.

2. The microneedle patch according to claim 1, wherein the nucleic acid drug is selected from a group consisting of plasmid DNA (pDNA), minicircular DNA (mcDNA), messenger RNA (mRNA), microRNA (miRNA), antisense oligonucleotides (ASOs), small interfering RNA (siRNA), aptamers, and ribozymes.

3. The microneedle patch according to claim 1, wherein the plurality of lipid nanoparticles are formed from at least a component selected from a group consisting of (4-hydroxybutyl) azanediyl bis(hexane-6,1-diyl)bis(2-hexyldecanoate), 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), 2,3-dioleyloxy-N-[2-(sperminecarboxamido)ethyl]-N,N-dimethyl-1-propanaminium trifluoroacetate (DOSPA), (2S)-2,5-bis(3-aminopropylamino)-N-[2-(dioctadecylamino)acetyl]pentanamide (DOGS; Transfectam), N1-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol59, N4-cholesteryl-spermine (GL67), (6Z,9Z,28Z,31Z)-heptatriaconta-6,9,28,31-tetraen-19-yl 4-(dimethylamino) butanoate (DLin-MC3-DMA; MC3), di((Z)-non-2-en-1-yl) 9-((4-(dimethylamino)butanoyl)oxy)heptadecanedioate (L319), biodegradable lipids heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102))78 and ((4-hydroxybutyl)azanediyl)bis(hexane-6,1-diyl)bis(2-hexyldecanoate) (ALC-0315), 3,6-bis(4-(bis(2-hydroxydodecyl)amino)butyl)piperazine-2,5-dione (cKK-E12), (2-hexyldecanoate), 2-[(polyethylene glycol)-2000]-N,N-ditetradecylacetamide, 1,2-dimyristoyl-rac-glycero-3-methoxypolyethylene glycol-2000 (PEG2000-DMG), 1,2-distearoyl-rac-glycero-3-methoxypolyethylene glycol-2000 (PEG2000-DSG), 1,2-distearoyl-sn-glycero-3-phosphoethanolamine-N-[methoxy(polyethylene glycol)-2000] (DSPE-PEG(2000)) 1,2-distearoyl-sn-glycero-3-phosphocholine (DSPC), 1,2-dioleoyl-sn-glycero-3-phosphoethanolamine (DOPE), Dipalmitoylphosphatidylcholine (DPPC), N-bis(2-hydroxyethyl)-N-methylethan-1-aminium bromide (BHEM-Cholesterol), and 3β-[N-(N′,N′-dimethylaminoethane)-carbamoyl]cholesterol (DC-Cholesterol).

4. The microneedle patch according to claim 1, wherein the lipid nanoparticles are formed from an ionizable cationic lipid, a polyethylene glycol (PEG), a cholesterol, and a helper lipid.

5. The microneedle patch according to claim 4, wherein the ionizable cationic lipid is 1,2-dioleoyl-3-trimethylammonium-propane (DOTAP).

6. The microneedle patch according to claim 4, wherein the polyethylene glycol (PEG) is 1,2-distearoyl-sn-glycero-3-phosphoethanolamine-N-[methoxy-(polyethylene glycol)-2000] (DSPE-PEG(2000)).

7. The microneedle patch according to claim 4, wherein the helper lipid is Dipalmitoylphosphatidylcholine (DPPC).

8. The microneedle patch according to claim 1, wherein the lipid nanoparticles are 50-220 nm or 100-150 nm in size.

9. The microneedle patch according to claim 1, wherein the lipid nanoparticles are 120±30 nm in size, with size distribution (PDI) less than 0.2.

10. The microneedle patch according to claim 1, wherein the one or more polymers are selected from a group comprising polyvinylpyrrolidone, polyvinyl alcohol, hyaluronic acid, collagen, polyethylene glycol, their derivatives or a mixture thereof.

11. The microneedle patch according to claim 1, wherein the one or more polymers is a polymer mixture of polyvinylpyrrolidone and polyvinyl alcohol.

12. The microneedle patch according to claim 11, wherein the polyvinylpyrrolidone is in a ratio of 0:100 to 100:0(%) against the polyvinyl alcohol.

13. The microneedle patch according to claim 11, wherein the polyvinylpyrrolidone has a molecular weight of 23000-32000 or 35000-51000 Daltons.

14. The microneedle patch according to claim 11, wherein the polyvinyl alcohol has a molecular weight of 72600-81400, 81400-94600 or 17600-26400 Daltons.

15. The microneedle patch according to claim 11, wherein the polymer mixture is further mixed with a sugar selected from a group consisting of sucrose, trehalose, dextrose and a mixture thereof to form a further mixture, and wherein the sugar has a concentration of 0.1-10% (w/w) in the further mixture.

16. The microneedle patch according to claim 1, wherein the nucleic acid drug is adapted to retain more than 50% of original activity in a sealed centrifuge tube at room temperature for a period of at least 42 days, and remains biologically active for at least 60 days.

17. A method of fabricating a microneedle patch, comprising steps of:
(i) forming a plurality of nanoparticles with a nucleic acid drug encapsulated therein,
(ii) mixing the plurality of nanoparticles with a polymeric solution to form a matrix solution, wherein the polymeric solution comprises one or more polymers dissolvable in human skin layer,
(iii) casting the matrix solution in a mold defined with a plurality of recesses for producing microneedle structures, wherein each of the microneedle structures is defined with a tip end portion that enables dermal penetration,
(iv) drying the matrix solution to form a microneedle patch storable in room temperature, and
(v) removing the microneedle patch from the mold.

18. The method according to claim 17, wherein the plurality of nanoparticles is formed by steps of:
(i) dissolving the at least one lipid component in a first solvent and the nucleic acid drug in a second solvent,
(ii) mixing the dissolved lipid component with the dissolved nucleic acid drug to form a drug mixture,
(iii) passing the drug mixture though both organic and aqueous phases to form nanosuspensions,
(iv) dialysing the nanosuspensions to remove the first and second solvents from the drug mixture, and
(v) centrifuging the nanosuspensions in a buffer solution to form the plurality of nanoparticles.

19. The method according to claim 18, wherein the first solvent is ethanol.

20. The method according to claim 18, wherein the second solvent is sodium citrate buffer solution.

21. The method according to claim 18, wherein the buffer solution is phosphate buffered saline solution.

22. The method according to claim 18, wherein the drug mixture is passed though the organic and aqueous phases at a flow rate of 1:3.

23. The method according to claim 18, wherein the drug mixture has a total flow rate of 4-6 ml/min between the aqueous and organic phases.

24. The method according to claim 18, wherein the drug mixture has a lipid concentration of 3-14 mg/ml.

25. The method according to claim 18, wherein the drug mixture is passed though the organic and aqueous phases in a microfluid chip, and wherein the microfluid chip is a Y-type or a T-type microfluidic chip.

26. The method according to claim 18, wherein the dialysing of the nanosuspensions is performed at a low temperature for 24 to 48 hours.

27. The method according to claim 26, wherein the low temperature is 4° C.

28. The method according to claim 17, wherein the polymeric solution has a concentration of 20-30%.

29. The method according to claim 17, wherein the mold is treated with plasma before the matrix solution is cast therein.

30. The method according to claim 17, further comprising a step of packing the microneedle patch in a nitrogen (N₂) environment.

31. The method according to claim 17, further comprising a step of storing the microneedle patch under temperature between 0-40° C. and humidity of or below about 40%.

32. The method according to claim 17, further comprising a step of storing the microneedle patch in a dry box at room temperature.

33. The method according to claim 17, wherein the mold is a PDMS mold, a metal mold, or a resin mold.

34. The method according to claim 17, wherein the nucleic acid drug is selected from a group consisting of plasmid DNA (pDNA), minicircular DNA (mcDNA), messenger RNA (mRNA), microRNA (miRNA), antisense oligonucleotides (ASOs), small interfering RNA (siRNA), aptamers, and ribozymes.

35. The method according to claim 17, wherein the plurality of lipid nanoparticles are formed from at least a component selected from a group consisting of (4-hydroxybutyl) azanediyl bis(hexane-6,1-diyl)bis(2-hexyldecanoate), 1,2-di-O-octadecenyl-3-trimethylammonium-propane (DOTMA), 1,2-dioleoyl-sn-glycero-3-phosphoethanolamine (DOPE), 1,2-dioleoyl-3-trimethylammonium-propane (DOTAP), Dimethyldioctadecylammonium bromide (DDAB), 2,3-dioleyloxy-N-[2-(sperminecarboxamido)ethyl]-N,N-dimethyl-1-propanaminium trifluoroacetate (DOSPA), (2S)-2,5-bis(3-aminopropylamino)-N-[2-(dioctadecylamino)acetyl]pentanamide (DOGS; Transfectam), N1-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol59, N4-cholesteryl-spermine (GL67), (6Z,9Z,28Z,31Z)-heptatriaconta-6,9,28,31-tetraen-19-yl 4-(dimethylamino) butanoate (DLin-MC3-DMA; MC3), di((Z)-non-2-en-1-yl) 9-((4-(dimethylamino)butanoyl)oxy)heptadecanedioate (L319), biodegradable lipids heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102))78 and ((4-hydroxybutyl)azanediyl)bis(hexane-6,1-diyl)bis(2-hexyldecanoate) (ALC-0315), 3,6-bis(4-(bis(2-hydroxydodecyl)amino)butyl)piperazine-2,5-dione (cKK-E12), (2-hexyldecanoate), 2-[(polyethylene glycol)-2000]-N,N-ditetradecylacetamide, 1,2-dimyristoyl-rac-glycero-3-methoxypolyethylene glycol-2000 (PEG2000-DMG), 1,2-distearoyl-rac-glycero-3-methoxypolyethylene glycol-2000 (PEG2000-DSG), 1,2-distearoyl-sn-glycero-3-phosphoethanolamine-N-[methoxy(polyethylene glycol)-2000] (DSPE-PEG(2000)) 1,2-distearoyl-sn-glycero-3-phosphocholine (DSPC), 1,2-dioleoyl-sn-glycero-3-phosphoethanolamine (DOPE), Dipalmitoylphosphatidylcholine (DPPC), N-bis(2-hydroxyethyl)-N-methylethan-1-aminium bromide (BHEM-Cholesterol), and 3β-[N-(N′,N′-dimethylaminoethane)-carbamoyl]cholesterol (DC-Cholesterol).

36. The method according to claim 17, wherein the lipid nanoparticles are formed from an ionizable cationic lipid, a polyethylene glycol (PEG), a cholesterol, and a helper lipid.

37. The method according to claim 36, wherein the ionizable cationic lipid is 1,2-dioleoyl-3-trimethylammonium-propane (DOTAP).

38. The method according to claim 36, wherein the polyethylene glycol (PEG) is 1,2-distearoyl-sn-glycero-3-phosphoethanolamine-N-[methoxy-(polyethylene glycol)-2000] (DSPE-PEG(2000)).

39. The method according to claim 36, wherein the helper lipid is Dipalmitoylphosphatidylcholine (DPPC).

40. The method according to claim 17, wherein the lipid nanoparticles are 50-220 nm or 100-150 nm in size.

41. The method according to claim 17, wherein the lipid nanoparticles are 120±30 nm in size, with size distribution (PDI) less than 0.2.

42. The method according to claim 17, wherein the one or more polymers are selected from a group comprising polyvinylpyrrolidone, polyvinyl alcohol, hyaluronic acid, collagen, polyethylene glycol, their derivatives or a mixture thereof.

43. The method according to claim 17, wherein the one or more polymers is a polymer mixture of polyvinylpyrrolidone and polyvinyl alcohol.

44. The method according to claim 17, wherein the polyvinylpyrrolidone is in a ratio of 0:100 to 100:0(%) against the polyvinyl alcohol.

45. The method according to claim 17, wherein the polyvinylpyrrolidone has a molecular weight of 23000-32000 or 35000-51000 Daltons.

46. The method according to claim 17, wherein the polyvinyl alcohol has a molecular weight of 72600-81400, 81400-94600 or 17600-26400 Daltons.

47. The method according to claim 17, further comprising a step of mixing the polymer mixture with a sugar selected from a group consisting of sucrose, trehalose, dextrose and a mixture thereof to form a further mixture, and wherein the sugar has a concentration of 0.1-10% (w/w) in the further mixture.

48. The method according to claim 17, wherein the nucleic acid drug is adapted to retain more than 50% of original activity in a sealed centrifuge tube at room temperature for a period of at least 42 days, and remains biologically active for at least 60 days.
